(19) United States
(12) Patent Application Publication
(10) Pub. No.: US 2002/0045578 A1
(43) Pub. Date: Apr. 18, 2002

(54) Solution structure of TNFR-1 DD and uses thereof

(76) Inventors: Steven F. Sukits, Arlington, MA (US); Guang-Yi Xu, Medford, MA (US); Lih-Ling Lin, Concord, MA (US); Jean-Baptiste Telliez, Waltham, MA (US); Sang Hsu, Lexington, MA (US)

Correspondence Address: Craig J. Arnold, Amster, Rothstein & Ebenstein, 90 Park Avenue, New York, NY, 10016, US

(21) Appl. No.: 09/854,906

(22) Filed: May 14, 2001

Related U.S. Application Data
(60) Provisional application No. 60/206,215, filed on May 22, 2000.

(51) Int. Cl. A61K 38/17; C07K 14/705

(52) U.S. Cl. 514/12; 530/350

(57) ABSTRACT

The present invention relates to the three dimensional solution structure of tumor necrosis factor receptor 1 death domain (TNFR-1 DD), as well as the identification and characterization of various binding active sites of TNFR-1 DD. Also provided for by the present invention are methods of utilizing the three dimensional structure for the design and selection of potent and selective inhibitors of TNF signaling pathways.

This application claims the benefit of U.S. Provisional Application No. 60/206,215 filed May 22, 2000.

### FIELD OF THE INVENTION

The present invention relates to the three dimensional solution structure of tumor necrosis factor receptor 1 death domain (TNFR-1 DD), as well as the identification of various active site structures of TNFR-1 DD. These structures are critical for the design and selection of potent and selective inhibitors of TNF signaling pathways.

### BACKGROUND OF THE INVENTION

Tumor Necrosis Factor Receptor 1 death domain (TNFR-1 DD) is the intracellular functional domain responsible for the receptor signaling activities. Tumor necrosis factor receptor-1, through trimerization induced by the binding of TNFα(cachectin) or TNFβ (lymphotoxin β) trimers, elicits a variety of biological responses including antiviral activity, cytotoxicity, and modulation of gene expression [1, 2]. TNFα and lymphotoxin α also bind and activate TNFR-2 [3]. Based on similarities in their cysteine-rich extracellular domains TNFR-1 and TNFR-2 belong to a receptor superfamily, which besides a number of death inducing receptors, includes CD40 and the low-affinity nerve growth factor receptor [4]. Although most cell types express both TNF receptors, TNFR-1 appears to play a predominant role in induction of gene expression and induction of cell death by TNFα [5, 6].

The death domain was originally described as a region of similarity within the intracellular portions of the TNFR-1 and Fas/Apo1 that is essential for the transduction of cytotoxic signals [7]. It was subsequently shown that the death domain is a protein interaction motif involved in homo- and hetero-association [8]. TNFR-1 trimerizes upon binding to the TNF trimer inducing association of its intracellular death domain [4]. The trimerization of TNFR-1 allows for the recruitment of an adaptor protein named TNFR-Associated Factor-2 (TRAF-2) [11] through interactions with the N-terminal domain of Fas Associated Death Domain (FADD) [11] through death domain interactions.

The solution structure of Fas, FADD and p75 neurotrophin receptor death domains have recently been determined by NMR spectroscopy [12-14]. The overall fold observed for these death domains consist of six antiparallel amphipathic α-helices packed into a globular structure. Two other domains called DED (Death effector domain) and CARD (Caspase recruitment domain) are structurally related to the death domain [15]. Recently the solution structure of the FADD-DED and RAIDD-CARD were solved revealing topologies very similar to the known death domain structures [16, 17]. Two recent studies have described the three-dimensional structure of Apaf-1 CARD in complex with the procaspase-9 CARD [18] and Tube death domain in complex with Pelle death domain [19]. These different studies illustrate the diverse association mechanisms in the death domain superfamily.

The low level of sequence conservation between death domains probably reflects their role in diverse cellular functions. Additionally, the apparent absence of a conserved interaction surface suggests that death domains may associate by a variety of mechanisms. Indeed, the surface formed by α-helices 2 and 3 has been implicated in the homo- and hetero-association of the death domains of FAS [12] and FADD [13]. Similar observations have been made in the case of the interaction of the CARD domains of Apaf-1 and procaspase-9 [18, 20-22]. Conversely, the complex formation of the Tube and Pelle death domains seems to rely on contacts between α-helices 4 and 5 of the Pelle death domain with α-helix 6 and the unique C-terminal tail of the Tube death domain [19]. The nature of the interaction also seems to vary between death domain complexes. Electrostatic interactions are thought to be a key component in the interaction between FAS death domain (FAS DD) and FADD death domain (FADD DD) [12]. Whereas, hydrogen bond contacts and van der Waals interactions have been shown to be involved in the complex of Tube and Pelle death domains [19], as well as in the complex of Apaf-1 and procaspase-9 CARD domains [18]. It is currently believed that the interactions between DEDs are hydrophobic in nature [16].

### SUMMARY OF THE INVENTION

The present invention relates to the three dimensional structure of a tumor necrosis factor receptor 1 death domain (TNFR-1 DD), and more specifically, to the solution structure of TNFR-1 DD, as determined using spectroscopy and various computer modeling techniques.

Particularly, the invention is further directed to the identification, characterization and three dimensional structure of an active site of TNFR-1 DD that provides an attractive target for the rational design of potent and selective inhibitors of TNF signaling pathways.

Accordingly, the present invention provides a solution comprising a tumor necrosis factor receptor 1 death domain (TNFR-1 DD). The three dimensional solution structure of TNFR-1 DD is provided by the relative atomic structural coordinates of FIG. 8, as obtained from spectroscopy data.

Also provided by the present invention is an active site of a TRADD DD binding protein or peptide, preferably of TNFR-1 DD, wherein said active site is characterized by a three dimensional structure comprising the relative structural coordinates of amino acid residues K343, E344, R347, R348 and D353 according to FIG. 8, ± a root mean square deviation from the conserved backbone atoms of said amino acids of not more than 1.5 Å.

Also provided for by the present invention is an active site of a TRADD DD binding protein or peptide, preferably of TNFR-1 DD, wherein said active site is characterized by a three dimensional structure comprising the relative structural coordinates of amino acid residues E369 and Y373 according to FIG. 8, ± a root mean square deviation from the conserved backbone atoms of said amino acids of not more than 1.5 Å.

In addition, the present invention provides an active site of a TRADD DD binding protein or peptide, preferably TNFR-1 DD, wherein said active site is characterized by a three dimensional structure comprising the relative structural coordinates of amino acid residues E335, E386, E390, D398, E406, D407, E409 and E410 according to FIG. 8, ± a root mean square deviation from the conserved backbone atoms of said amino acids of not more than 1.5 Å.

Still further, the present invention provides an active site of a TRADD DD binding protein or peptide, preferably TNFR-1 DD, wherein said active site is characterized by a three dimensional structure comprising the relative structural coordinates of amino acid residues R358, R365, R368, R379, R380, R381, R384, R385, R394 and R397 according to FIG. 8, ± a root mean square deviation from the conserved backbone atoms of said amino acids of not more than 1.5 Å.

The solution coordinates of TNFR-1 DD or portions thereof (such as the TRADD DD binding site or other active sites), as provided by this invention may be stored in a machine-readable form on a machine-readable storage medium, e.g. a computer hard drive, diskette, DAT tape, etc., for display as a three-dimensional shape or for other uses involving computer-assisted manipulation of, or computation based on, the structural coordinates or the three-dimensional structures they define. By way of example, the data defining the three dimensional structure of TNFR-1 DD as set forth in FIG. 8 may be stored in a machine-readable storage medium, and may be displayed as a graphical three-dimensional representation of the relevant structural coordinates, typically using a computer capable of reading the data from said storage medium and programmed with instructions for creating the representation from such data.

Accordingly, the present invention provides a machine, such as a computer, programmed in memory with the coordinates of TNFR-1 DD or portions thereof, together with a program capable of converting the coordinates into a three dimensional graphical representation of the structural coordinates on a display connected to the machine. A machine having a memory containing such data aids in the rational design or selection of inhibitors of TNFR-1 DD binding or activity, including the evaluation of the ability of a particular chemical entity to favorably associate with TNFR-1 DD as disclosed herein, as well as in the modeling of compounds, proteins, complexes, etc. related by structural or sequence homology to TNFR-1 DD.

The present invention is additionally directed to a method of determining the three dimensional structure of a molecule or molecular complex whose structure is unknown, comprising the steps of first obtaining crystals or a solution of the molecule or molecular complex whose structure is unknown, and then generating X-ray diffraction data from the crystallized molecule or molecular complex and/or generating NMR data from the solution of the molecule or molecular complex. The generated diffraction or spectroscopy data from the molecule or molecular complex can then be compared with the solution coordinates or three dimensional structure of TNFR-1 DD as disclosed herein, and the three dimensional structure of the unknown molecule or molecular complex conformed to the TNFR-1 DD structure using standard techniques such as molecular replacement analysis, 2D, 3D and 4D isotope filtering, editing and triple resonance NMR techniques, and computer homology modeling. Alternatively, a three dimensional model of the unknown molecule may be generated by generating a sequence alignment between TNFR-1 DD and the unknown molecule, based on any or all of amino acid sequence identity, secondary structure elements or tertiary folds, and then generating by computer modeling a three dimensional structure for the molecule using the three dimensional structure of, and sequence alignment with, TNFR-1 DD.

The present invention further provides a method for identifying an agent that interacts with TNFR-1 DD, comprising the steps of determining an active site of TNFR-1 DD using the three dimensional TNFR-1 DD structure, and then performing computer fitting analyses to identify an agent which interacts with the identified active site. Once the agent has been identified, it may be contacted with TNFR-1 DD (using TNFR-1 DD or a molecule comprising TNFR-1 DD such as TNFR-1), and the effect the agent has on TNFR-1 DD may then be assessed. In addition, the agent may be contacted with TNFR-1 DD (using TNFR-1 DD or a molecule comprising TNFR-1 DD such as TNFR-1) in the presence of a TNFR-1 DD binding molecule (including but not limited to TRADD DD), and the effect the agent has on binding between TNFR-1 DD and the TNFR-1 DD binding molecule may then be assessed.

Also provided is a method for identifying a potential inhibitor of TNFR-1 DD, comprising the steps of using a three dimensional structure of TNFR-1 DD as defined by the relative structural coordinates of amino acids encoding TNFR-1 DD to design or select a potential inhibitor, and obtaining or synthesizing said potential inhibitor. The inhibitor may be selected by screening an appropriate database, may designed de novo by analyzing the steric configurations and charge potentials of an empty TNFR-1 DD active site in conjunction with the appropriate software programs, or may be designed using characteristics of known inhibitors to create “hybrid” inhibitors. The inhibitor may then be contacted with TNFR-1 DD alone (using TNFR-1 DD or a molecule comprising TNFR-1 DD such as TNFR-1), or in the presence of a TNFR-1 DD binding molecule such as TRADD DD, and the effect of the inhibitor on TNFR-1 alone or binding between TNFR-1 and the TNFR-1 DD binding molecule may be assessed. It is also within the confines of the present invention that a potential inhibitor may be designed or selected by identifying chemical entities or fragments capable of associating with TNFR-1 DD; and assembling the identified chemical entities or fragments into a single molecule to provide the structure of the potential inhibitor.

Finally, the present invention provides agents or inhibitors designed or selected using the methods disclosed herein. Additional objects of the present invention will be apparent from the description which follows.

### BRIEF DESCRIPTION OF THE FIGURES

FIG. 1 is a summary of NMR-derived secondary structure indicators measured for TNFR-DD R347A together with the deduced secondary structure. For NOE data, the thickness of the lines reflects strength of sequential NOEs.

FIG. 2 is a summary of the inter-helical NOE contacts of TNFR-DD R347A at pH8.8. The helices 1, 3 and 5 are viewed from their N-termini, while helices 2, 4 and 6 are viewed from their C-termini. The bold and boxed residues have altered binding properties when mutated to alanine (see text and FIG. 5). The bold and italic residues have unaltered binding properties when mutated to alanine (see text and FIG. 5).

FIG. 3 depicts the effect of the ionic strength on the binding of TNFR-DD (200ng/ml) to MBP-TNFR-DD (left bar), MBP-TRADD (right bar).

FIG. 4 represents a sequence alignment of the death domains of TNFR-1 and Fas. The helices in the NMR structure of the TNFR-1 and Fas (Huang et al., 1996) are indicated. The mutations are indicated by an arrow pointing to the amino acid of substitution. A minus sign above the TNFR-1 mutation indicates a loss in self-association or interaction with TRADD-DD (see text). A minus sign below the Fas mutation indicates that this mutant aggregates less than the wild-type protein (Huang et al., 1996).

FIGS. 5A and 5B depict an ELISA of TNFR-DD wild-type and mutants binding to MBP-TNFR-DD and MBP-TRADD. FIG. 5A: Binding of the TNFR-DD mutants to MBP-TNFR-DD immobilized on the plate at 200 ng/well. The reference at 100% is the binding of the wild-type protein to MBP-TNFR-DD. The values used for the determination of the binding percentage were obtained with ˜300 ng/ml of soluble protein. FIG. 5B: Binding of the TNFR-DD mutants to MBP-TRADD. The experimental conditions used were the same as with MBP-TNFR-DD.

FIG. 6 depicts a summary of H-D exchange, ³J_{HNHα} scalar coupling information, sequential NOEs (d_{αN}, d_{NN}, d_{βN}), medium range NOEs (d_{NN(i.i+2)}, d_{αN(i.i+3))}, d_{αN(i.i+4)}, d_{αβ(i.i+3)}), and the Δ¹³C_{α} and Δ¹³C_{β}. Amides that didn't exchange with D₂O within 30 minutes are designated with an (•). The residues having a ³J_{HNHα} less than 5 Hz are designated with an solid line. The intensities of the NOEs are represented by the thickness of the lines. The values of the Δ¹³C_{α} and Δ¹³C₆₂ are represented by the intensity of the blocks. The secondary structure highlighting the 6 α-helices is indicated at the bottom.

FIG. 7 depicts the secondary structure and sequence alignment of TNFR-1 DD with proteins in the death domain superfamily. In instances when the lengths of the helices differed when compared to TNFR-1 DD, the sequences were aligned based upon sequence homology instead of secondary structure alignment.

FIG. 8 lists the atomic structure coordinates for the restrained minimized mean structure of TNFR-1 DD as derived by multidimensional NMR spectroscopy. “Atom type” refers to the atom whose coordinates are being measured. “Residue” refers to the type of residue of which each measured atom is a part—i.e., amino acid, cofactor, ligand or solvent. The “x, y and z” coordinates indicate the Cartesian coordinates of each measured atom's location (Å). All non-protein atoms are listed as HETATM instead of atoms using PDB conventions.

### DETAILED DESCRIPTION OF THE INVENTION

As used herein, the following terms and phrases shall have the meanings set forth below:

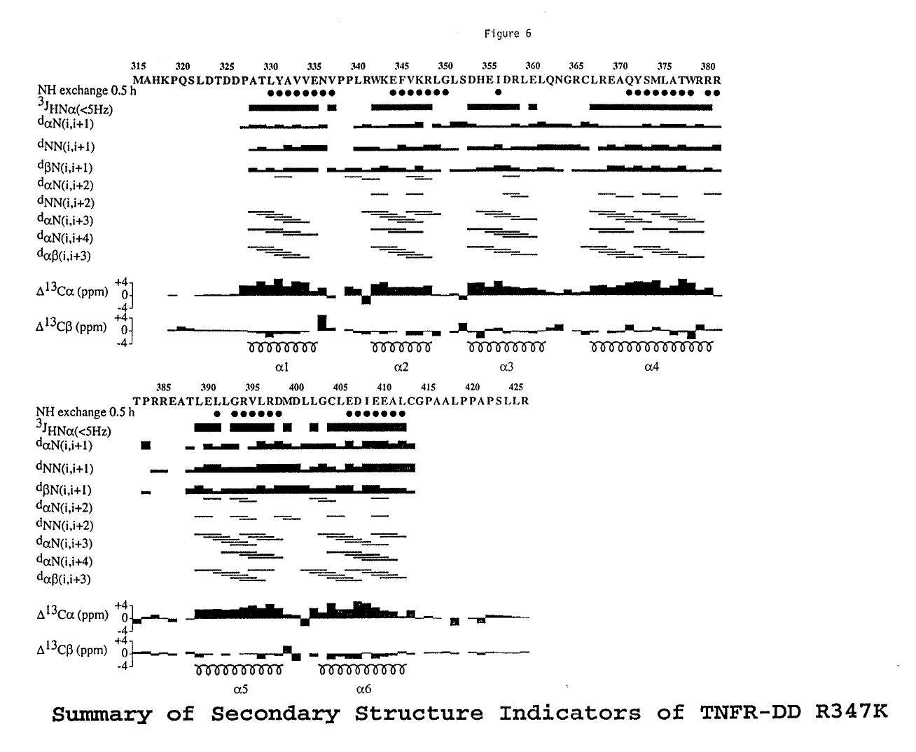

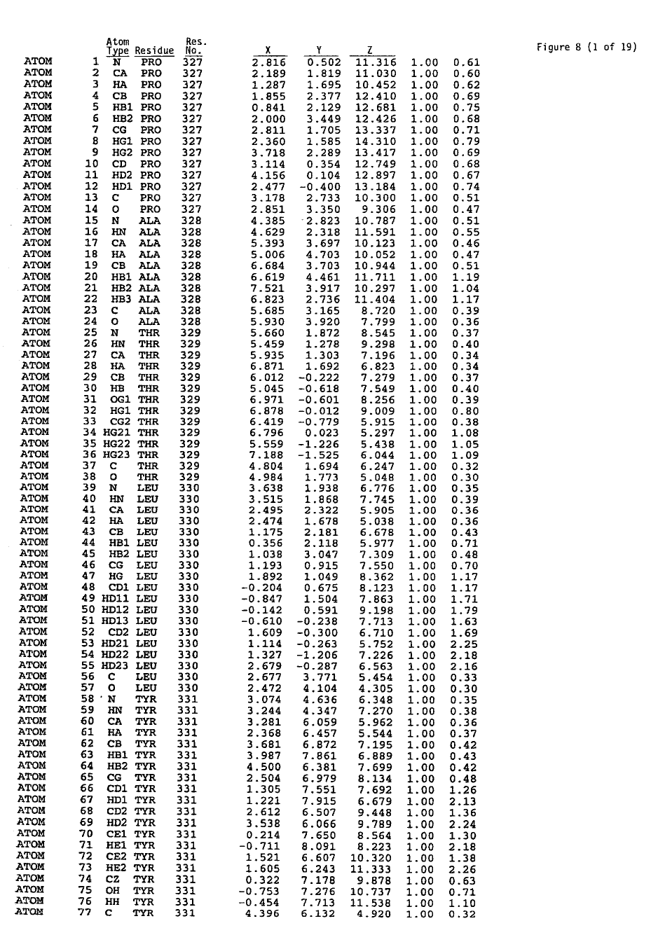

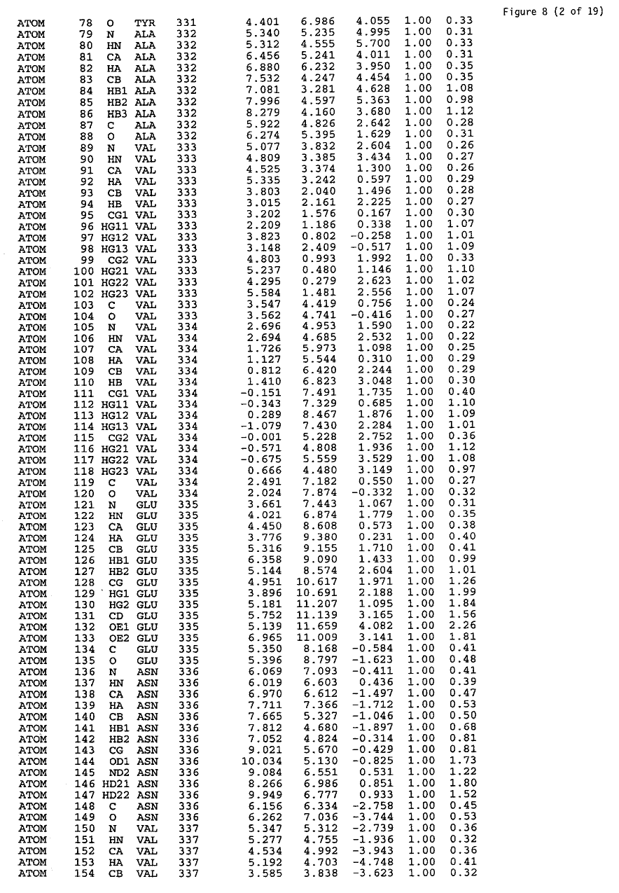

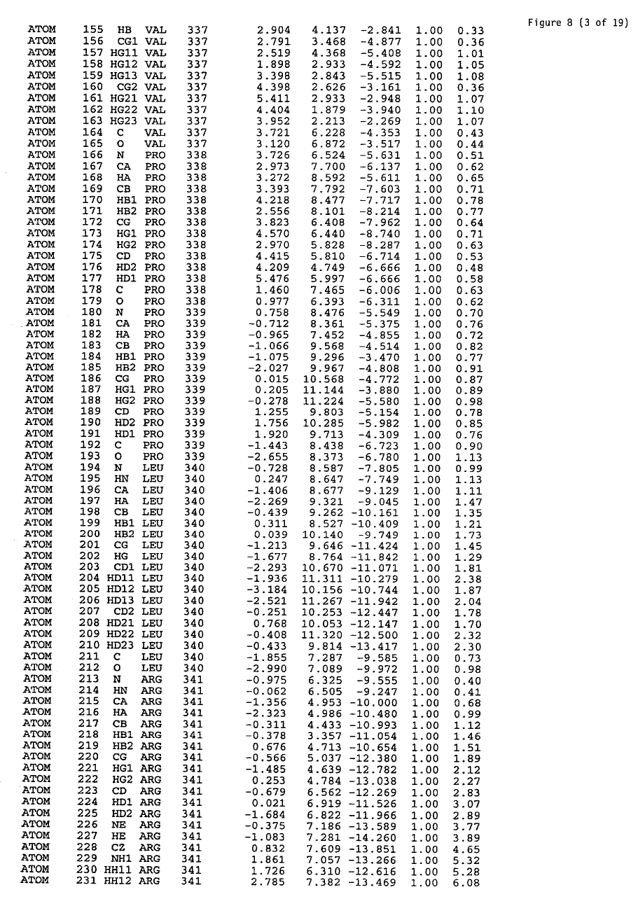

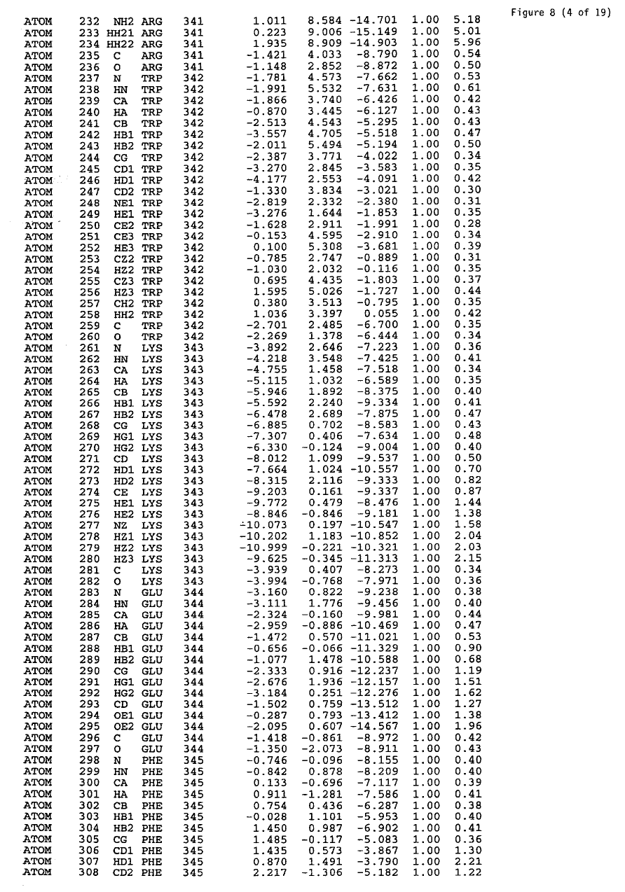

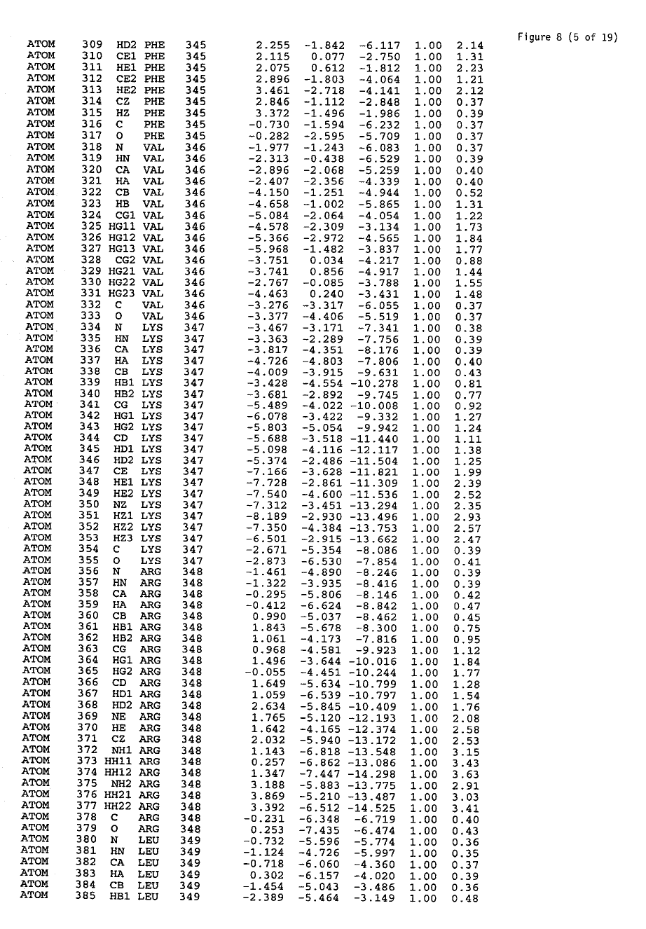

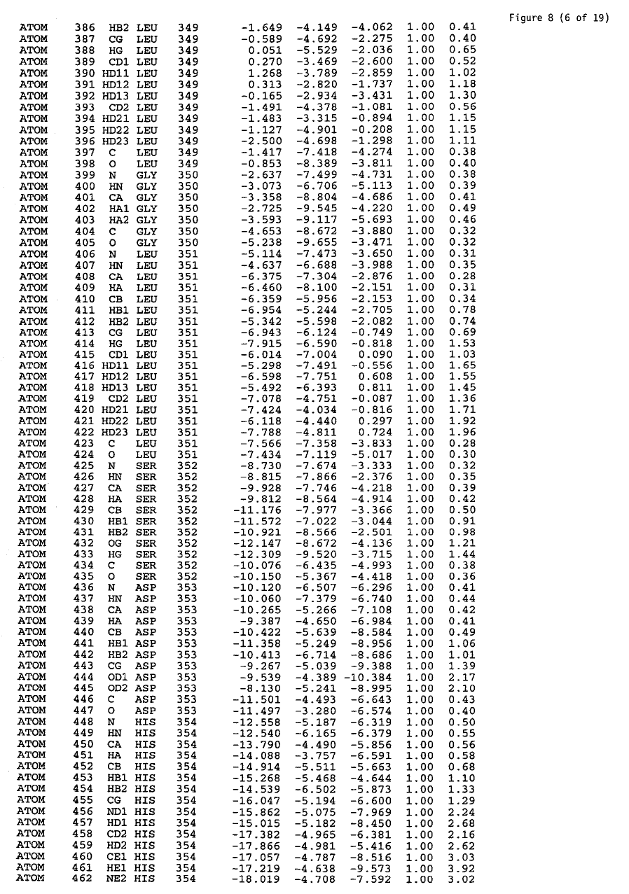

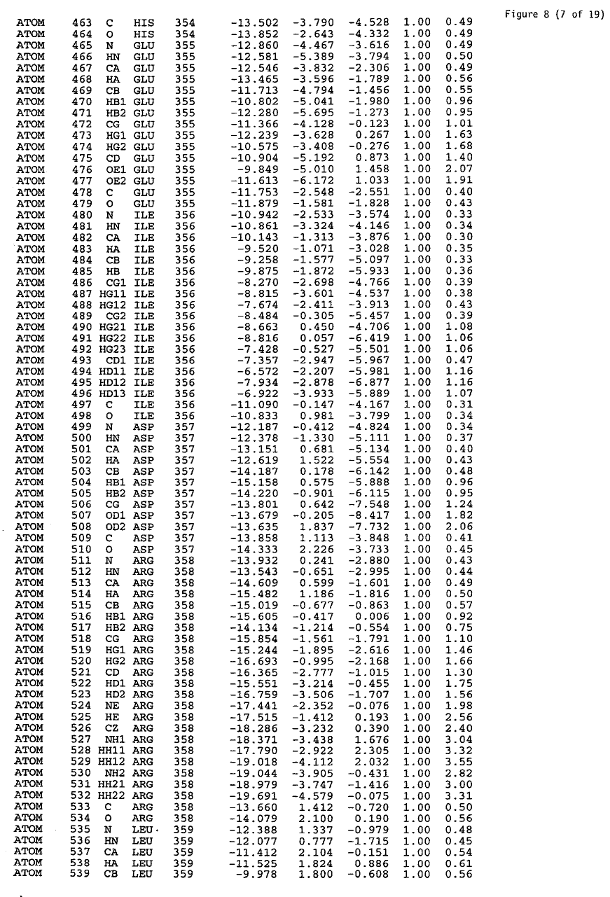

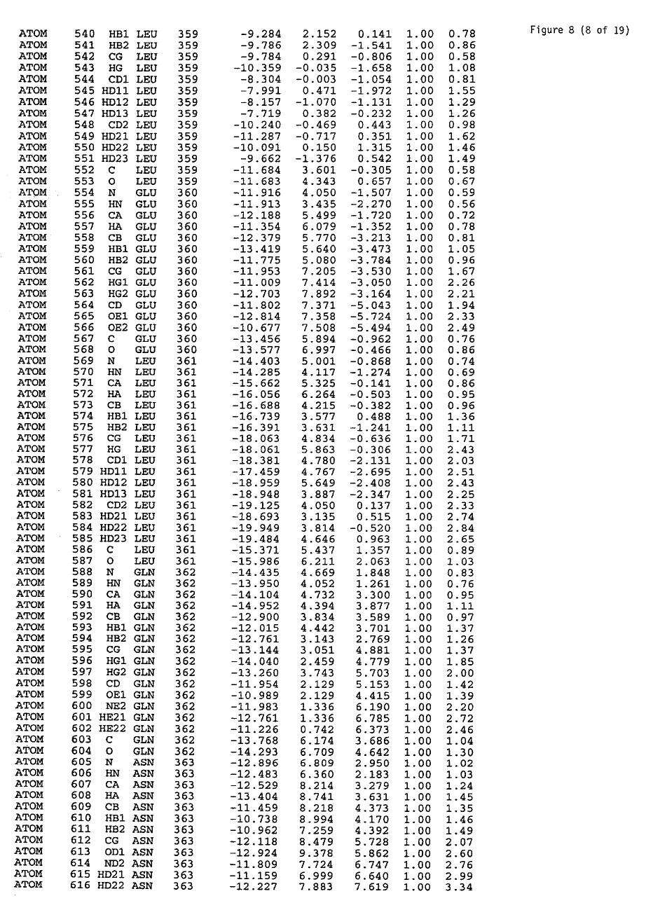

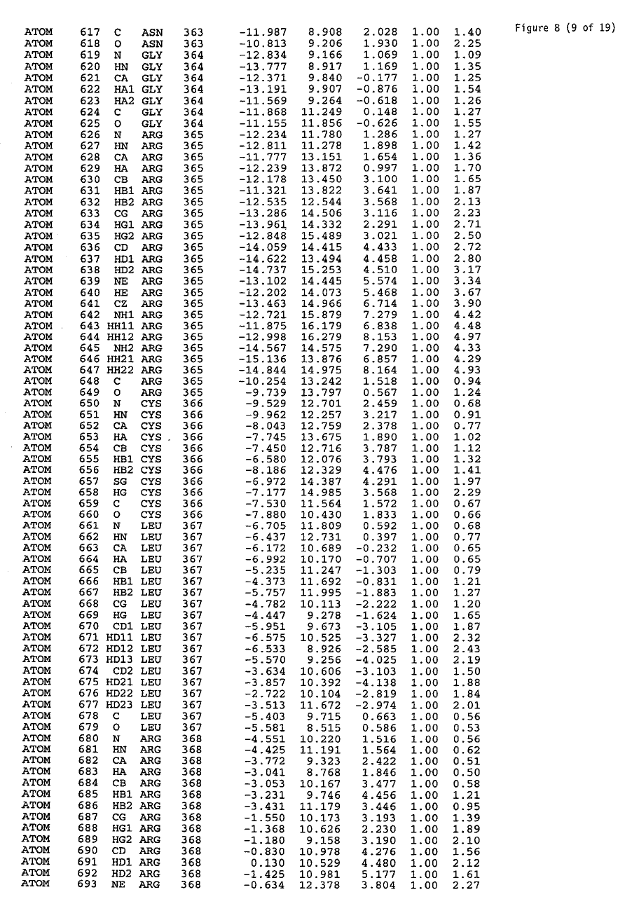

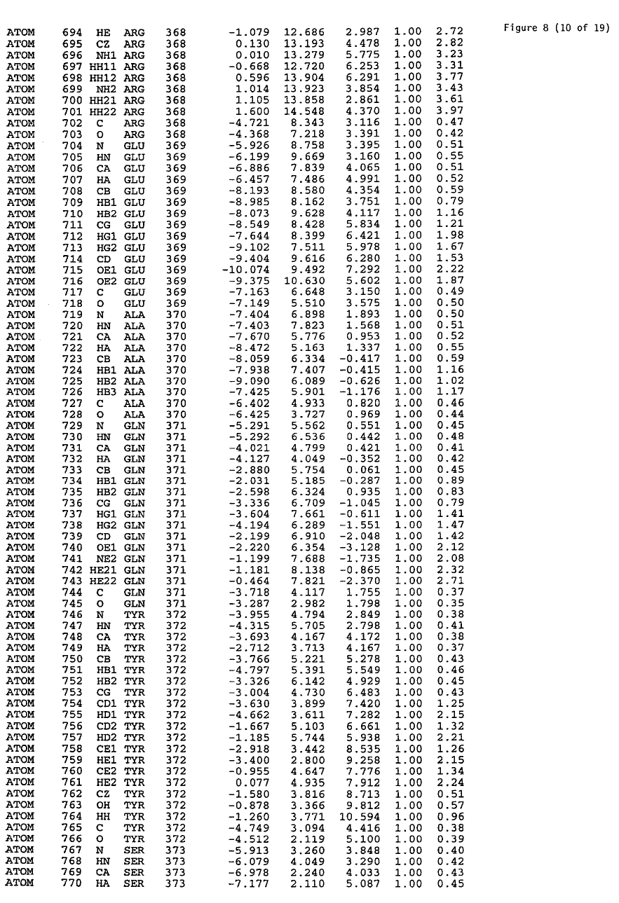

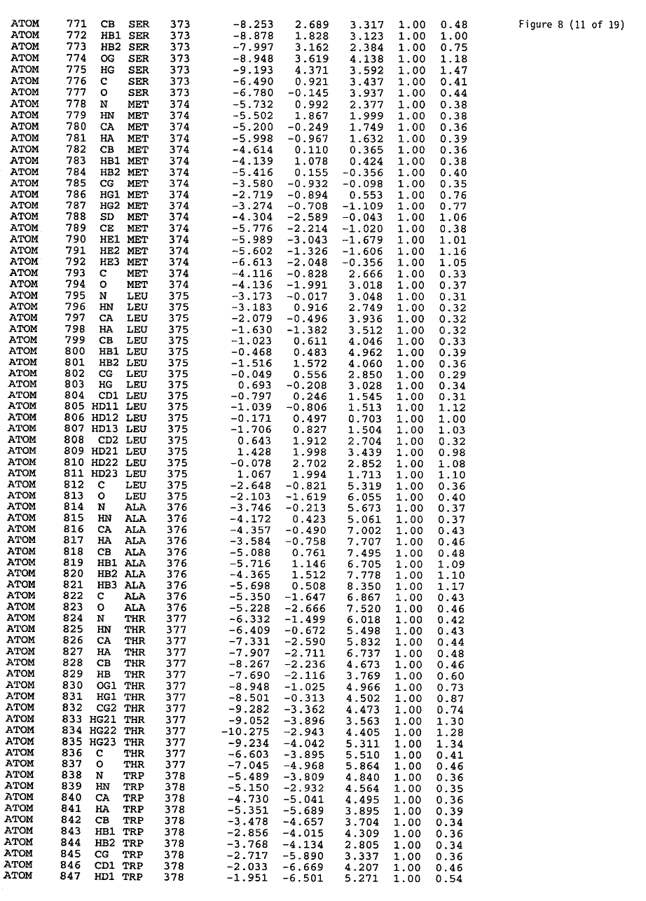

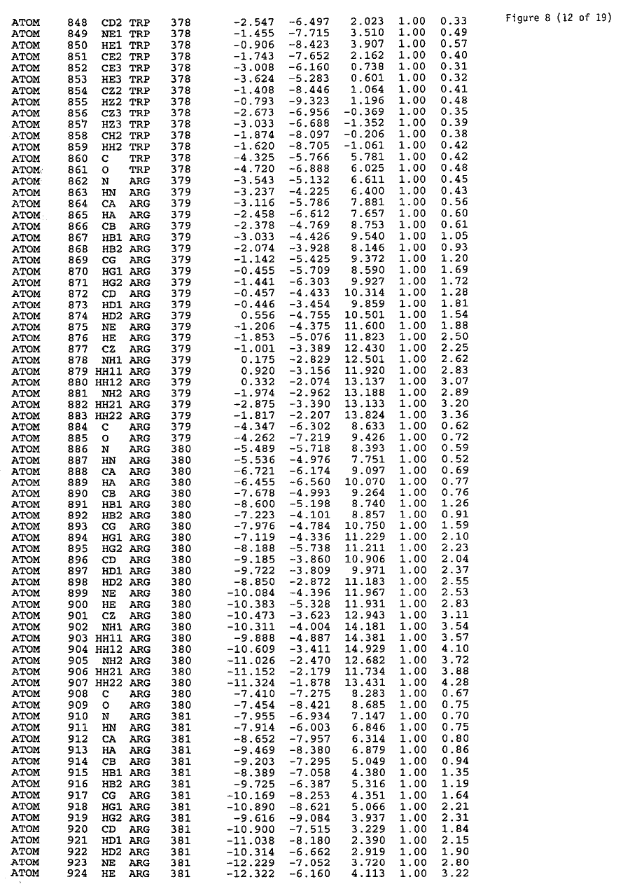

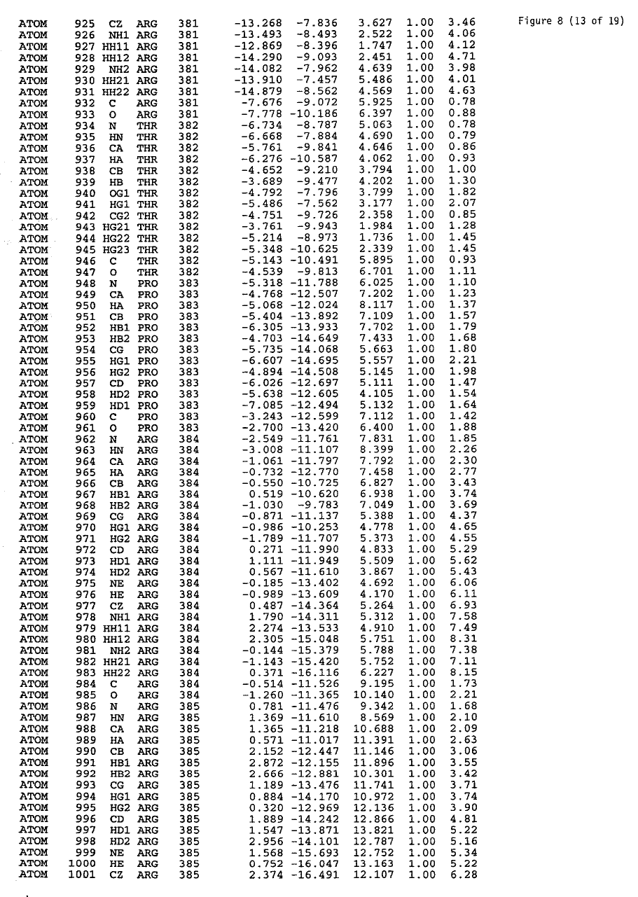

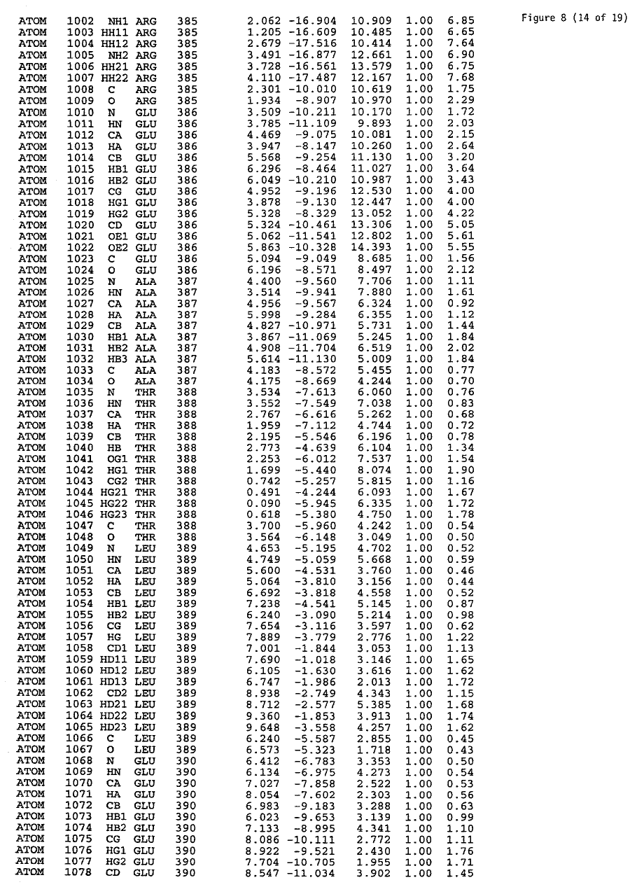

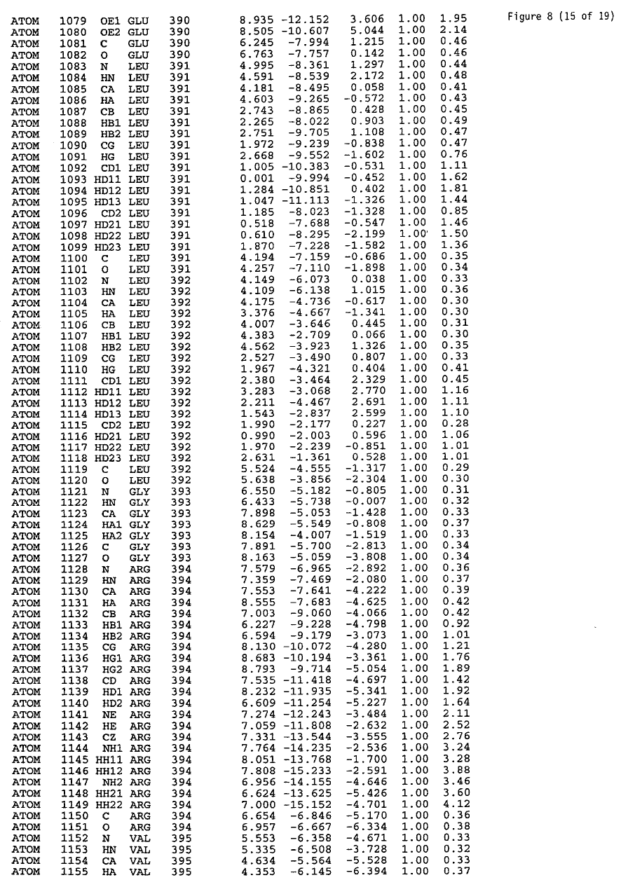

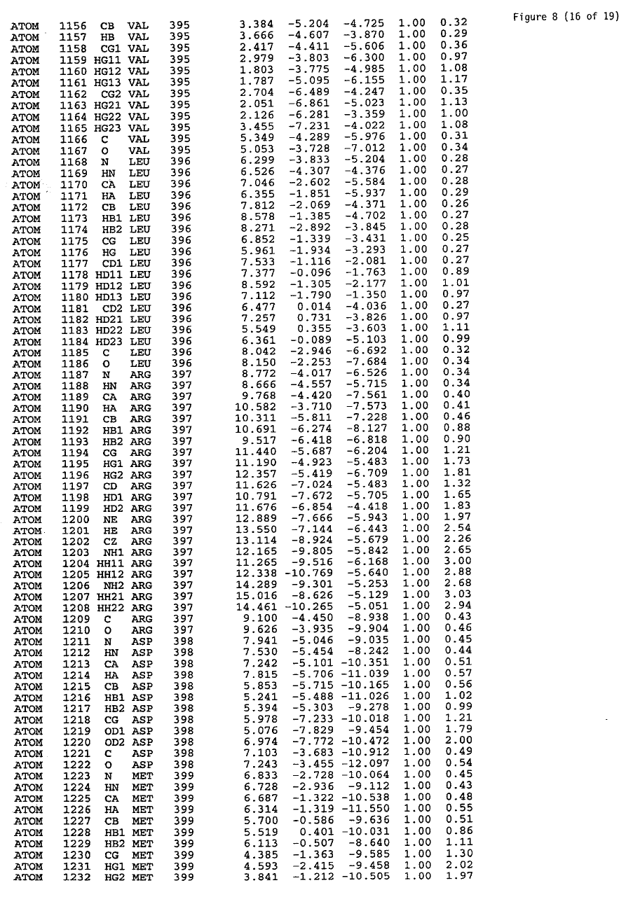

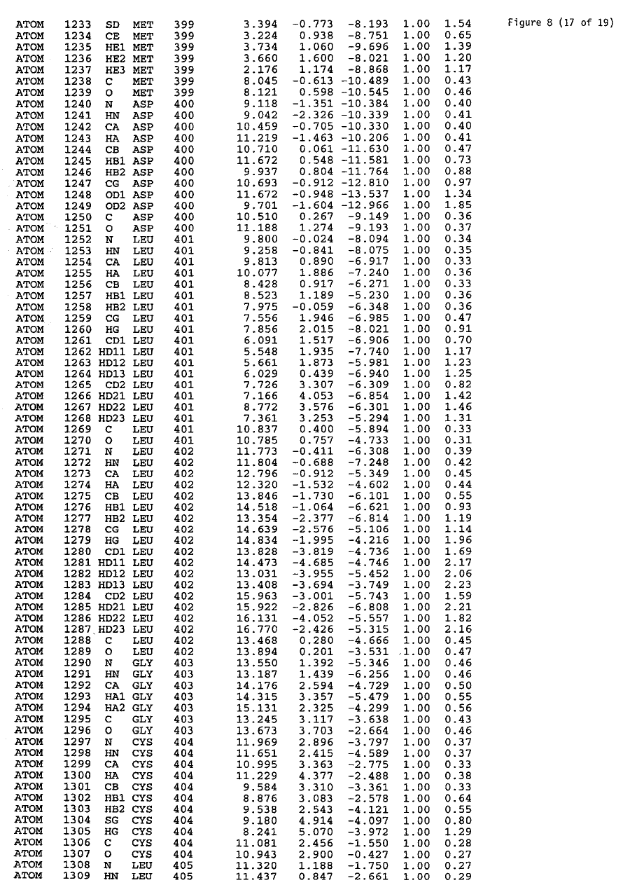

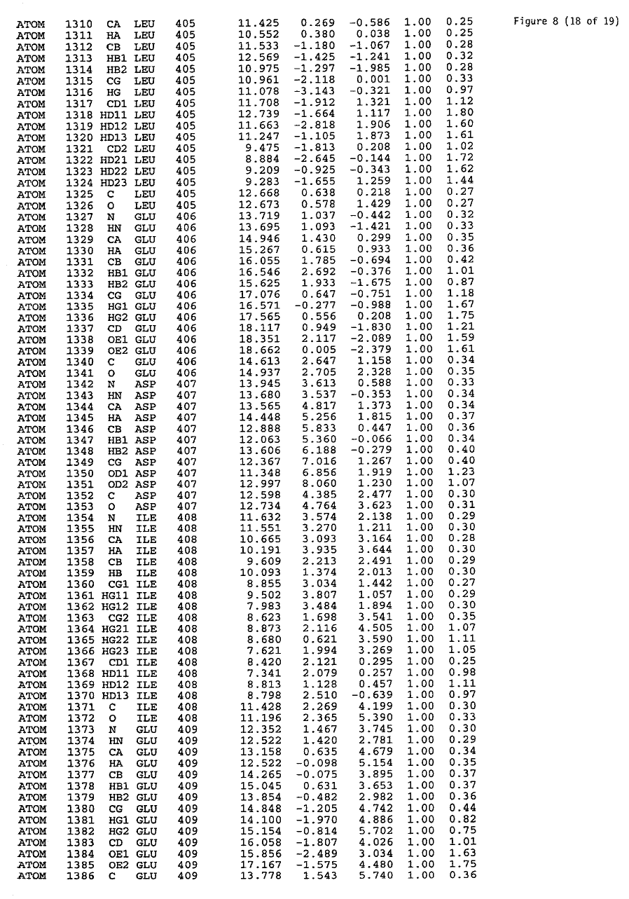

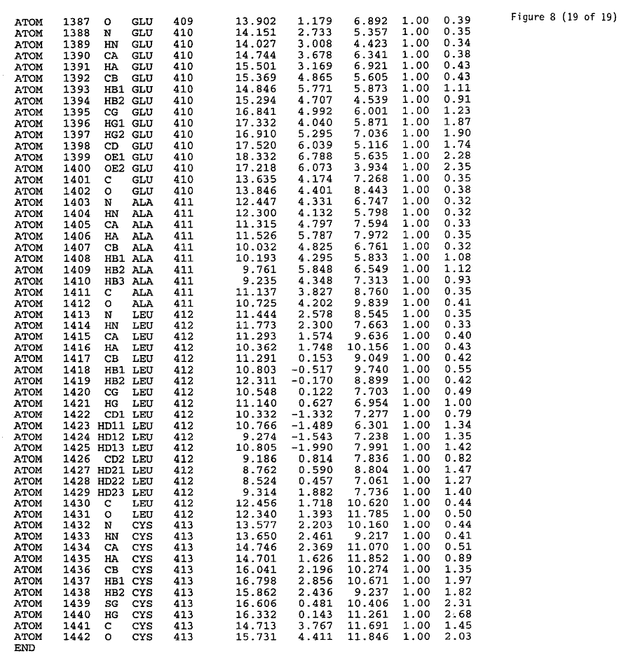

Unless otherwise noted, “TNFR-1 DD” includes both the death domain of TNFR-1 as encoded by the amino acid sequence of FIG. 6 (including conservative substitutions thereof), as well as “TNFR-1 DD analogues”, defined herein as proteins or peptides comprising a TRADD DD or TRADD DD-like binding site. Such TNFR-1 DD analogues include, but are not limited to, an active site characterized by a three dimensional structure comprising (i) the relative structural coordinates of amino acid residues K343, E344, R347, R348 and D353 according to FIG. 8, (ii) the relative structural coordinates of amino acid residues E369 and Y373 according to FIG. 8, (iii) the relative structural coordinates of amino acid residues E335, E386, E390, D398, E406, D407, E409 and E410 according to FIG. 8, or (iv) the relative structural coordinates of amino acid residues R358, R365, R368, R379, R380, R381, R384, R385, R394 and R397 according to FIG. 8, in each case, ± a root mean square deviation from the conserved backbone atoms of said amino acids of not more than 1.5 Å, more preferably not more than 1.0 Å, and most preferably not more than 0.5 Å.

Unless otherwise indicated, “protein” or “molecule” shall include a protein, protein domain, polypeptide or peptide.

“Structural coordinates” are the Cartesian coordinates corresponding to an atom's spatial relationship to other atoms in a molecule or molecular complex. Structural coordinates may be obtained using x-ray crystallography techniques or NMR techniques, or may be derived using molecular replacement analysis or homology modeling. Various software programs allow for the graphical representation of a set of structural coordinates to obtain a three dimensional representation of a molecule or molecular complex. The structural coordinates of the present invention may be modified from the original set provided in FIG. 8 by mathematical manipulation, such as by inversion or integer additions or subtractions. As such, it is recognized that the structural coordinates of the present invention are relative, and are in no way specifically limited by the actual x, y, z coordinates of FIG. 8.

An “agent” shall include a protein, polypeptide, peptide, nucleic acid, including DNA or RNA, molecule, compound or drug.

“Root mean square deviation” is the square root of the arithmetic mean of the squares of the deviations from the mean, and is a way of expressing deviation or variation from the structural coordinates described herein. The present invention includes all embodiments comprising conservative substitutions of the noted amino acid residues resulting in same structural coordinates within the stated root mean square deviation.

It will be obvious to the skilled practitioner that the numbering of the amino acid residues in the various isoforms of TNFR-1 DD covered by the present invention may be different than that set forth herein, or may contain certain conservative amino acid substitutions that yield the same three dimensional or solution structures as those defined by FIG. 8 herein. Corresponding amino acids and conservative substitutions in other isoforms or analogues are easily identified by visual inspection of the relevant amino acid sequences or by using commercially available homology software programs.

“Conservative substitutions” are those amino acid substitutions which are functionally equivalent to the substituted amino acid residue, either by way of having similar polarity, steric arrangement, or by belonging to the same class as the substituted residue (e.g., hydrophobic, acidic or basic), and includes substitutions having an inconsequential effect on the three dimensional structure of TNFR-1 DD with respect to the use of said structure for the identification and design of TNFR-1 DD or TNFR-1 DD complex inhibitors, for molecular replacement analyses and/or for homology modeling.

An “active site” refers to a region of a molecule or molecular complex that, as a result of its shape and charge potential, favorably interacts or associates with another agent (including, without limitation, a protein, polypeptide, peptide, nucleic acid, including DNA or RNA, molecule, compound, antibiotic or drug) via various covalent and/or non-covalent binding forces. As such, an active site of the present invention may include, for example, the actual site of TRADD DD binding with TNFR-1 DD, as well as accessory binding sites adjacent to the actual site of TRADD DD binding that nonetheless may affect TNFR-1 DD upon interaction or association with a particular agent, either by direct interference with the actual site of TRADD DD binding or by indirectly affecting the steric conformation or charge potential of TNFR-1 DD and thereby preventing or reducing TRADD DD binding to TNFR-1 DD at the actual site of TRADD DD binding. As used herein, “active site” also includes the TNFR-1 DD site of self association, as well as other binding sites present on TNFR-1 DD.

A “TNFR-1 DD complex” refers to a co-complex of a molecule comprising the TNFR-1 DD region in bound association with a protein, polypeptide, peptide, nucleic acid, including DNA or RNA, small molecule, compound or drug, either by covalent or non-covalent binding forces. A non-limiting example of a TNFR-1 DD complex includes TNFR-1, TNFR-1 DD or a TNFR-1 DD analogue bound to TRADD DD.

The present invention relates to the three dimensional structure of TNFR-1 DD or of a TNFR-1 DD analogue, and more specifically, to the solution structure of TNFR-1 DD as determined using multidimensional NMR spectroscopy and various computer modeling techniques. The solution coordinates of TNFR-1 DD (disclosed herein at FIG. 8) are useful for a number of applications, including, but not limited to, the characterization of a three dimensional structure of TNFR-1 DD, as well as the visualization, identification and characterization of TNFR-1 DD active sites, including the site of TRADD DD binding to TNFR-1 DD. The active site structures may then be used to predict the orientation and binding affinity of a designed or selected inhibitor of TNFR-1 DD, a TNFR-1 DD analogue or of a TNFR-1 DD complex. Accordingly, the invention is particularly directed to the three dimensional structure of a TNFR-1 DD active site, including but not limited to the TRADD DD binding site.

As used herein, the TNFR-1 DD in solution comprises amino acid residues 316-425 of TNFR-1, and more preferably, the amino acid residues 316-425 set forth in FIG. 6, or conservative substitutions thereof. Preferably, the TNFR-1 DD in solution is either unlabeled, ¹⁵N enriched or ¹⁵N,¹³C enriched, and is preferably biologically active. In addition, the secondary structure of the TNFR-1 DD in the solutions of the present invention comprises six alpha (α) helices. In this regard, α1 comprises amino acid residues A328-N336 of TNFR-1 DD, α2 comprises amino acid residues W342-L349 of TNFR-1 DD, α3 comprises amino acid residues P353-L361 of TNFR-1 DD, α4 comprises amino acid residues L367-R380 of TNFR-1 DD, α5 comprises amino acid residues L389-D398 of TNFR-1 DD and α6 comprises amino acid residues G403-L412 of TNFR-1 DD.

The protein or peptide used in the solution of the present invention includes TNFR-1 DD, as well as TNFR-1 DD analogues, where said protein or peptide preferably comprises an active site characterized by a three dimensional structure comprising (i) the relative structural coordinates of amino acid residues K343, E344, R347, R348 and D353 according to FIG. 8, (ii) the relative structural coordinates of amino acid residues E369 and Y373 according to FIG. 8, (iii) the relative structural coordinates of amino acid residues E335, E386, E390, D398, E406, D407, E409 and E410 according to FIG. 8, or (iv) the relative structural coordinates of amino acid residues R358, R365, R368, R379, R380, R381, R384, R385, R394 and R397 according to FIG. 8, in each case, ± a root mean square deviation from the conserved backbone atoms of said amino acids of not more than 1.5 Å, more preferably not more than 1.0 Å, and most preferably not more than 0.5 Å. In the most preferred embodiment, the protein or peptide used in the solution of the present invention is characterized by a three dimensional structure comprising the complete structural coordinates of the amino acids according to FIG. 8, ± a root mean square deviation from the conserved backbone atoms of said amino acids of not more than 1.5 Å (or more preferably, not more than 1.0 Å, and most preferably, not more than 0.5 Å).

Molecular modeling methods known in the art may be used to identify an active site or binding pocket of TNFR-1 DD, a TNFR-1 DD complex, or of a TNFR-1 DD analogue. Specifically, the solution structural coordinates provided by the present invention may be used to characterize a three dimensional structure of the TNFR-1 DD molecule, molecular complex or TNFR-1 DD analogue. From such a structure, putative active sites may be computationally visualized, identified and characterized based on the surface structure of the molecule, surface charge, steric arrangement, the presence of reactive amino acids, regions of hydrophobicity or hydrophilicity, etc. Such putative active sites may be further refined using chemical shift perturbations of spectra generated from various and distinct TNFR-1 DD complexes, competitive and non-competitive inhibition experiments, and/or by the generation and characterization of TNFR-1 DD or ligand mutants to identify critical residues or characteristics of the active site.

The identification of putative active sites of a molecule or molecular complex is of great importance, as most often the biological activity of a molecule or molecular complex results from the interaction between an agent and one or more active sites of the molecule or molecular complex. Accordingly, the active sites of a molecule or molecular complex are the best targets to use in the design or selection of inhibitors that affect the activity of the molecule or molecular complex.

The present invention is directed to an active site of TNFR 1 DD, a TNFR-1 DD complex or of a TNFR-1 DD analogue, that, as a result of its shape, reactivity, charge potential, etc., favorably interacts or associates with another agent (including, without limitation, a protein, polypeptide, peptide, nucleic acid, including DNA or RNA, molecule, compound, antibiotic or drug). Preferably, the present invention is directed to an active site of a TRADD DD binding protein or peptide, and preferably TNFR-1 DD, that is characterized by the three dimensional structure comprising the relative structural coordinates of amino acid residues K343, E344, R347, R348 and D353 according to FIG. 8, ± a root mean square deviation from the conserved backbone atoms of said amino acids of not more than 1.5 Å, preferably not more than 1.0 Å, and most preferably not more than 0.5 Å. In another embodiment, the active site of a TRADD DD binding protein or peptide, and preferably TNFR-1 DD, is characterized by the three dimensional structure comprising the relative structural coordinates of amino acid residues E369 and Y373 according to FIG. 8, ± a root mean square deviation from the conserved backbone atoms of said amino acids of not more than 1.5 Å, preferably not more than 1.0 Å, and most preferably not more than 0.5 Å. In another embodiment, the active site of a TRADD DD binding protein or peptide, and preferably TNFR-1 DD, is characterized by the three dimensional structure comprising the relative structural coordinates of amino acid residues E335, E386, E390, D398, E406, D407, E409 and E410 according to FIG. 8, ± a root mean square deviation from the conserved backbone atoms of said amino acids of not more than 1.5 Å, preferably not more than 1.0 Å, and most preferably not more than 0.5 Å. In yet another embodiment, the active site of a TRADD DD binding protein or peptide, and preferably TNFR-1 DD, is characterized by the three dimensional structure comprising the relative structural coordinates of amino acid residues R358, R365, R368, R379, R380, R381, R384, R385, R394 and R397 according to FIG. 8, ± a root mean square deviation from the conserved backbone atoms of said amino acids of not more than 1.5 Å, preferably not more than 1.0 Å, and most preferably not more than 0.5 Å.

In order to use the structural coordinates generated for a solution structure of the present invention as set forth in FIG. 8, it is often necessary to display the relevant coordinates as, or convert them to, a three dimensional shape or graphical representation, or to otherwise manipulate them. For example, a three dimensional representation of the structural coordinates is often used in rational drug design, molecular replacement analysis, homology modeling, and mutation analysis. This is typically accomplished using any of a wide variety of commercially available software programs capable of generating three dimensional graphical representations of molecules or portions thereof from a set of structural coordinates. Examples of said commercially available software programs include, without limitation, the following: GRID (Oxford University, Oxford, UK); MCSS (Molecular Simulations, San Diego, Calif.); AUTODOCK (Scripps Research Institute, La Jolla, Calif.); DOCK (University of California, San Francisco, Calif.); Flo99 (Thistlesoft, Morris Township, N.J.); Ludi (Molecular Simulations, San Diego, Calif.); QUANTA (Molecular Simulations, San Diego, Calif.); Insight (Molecular Simulations, San Diego, Calif.); SYBYL (TRIPOS, Inc., St. Louis, Mo.); and LEAPFROG (TRIPOS, Inc., St. Louis, Mo.).

For storage, transfer and use with such programs, a machine, such as a computer, is provided for that produces a three dimensional representation of the TNFR-1 DD, a portion thereof (such as an active site or a binding site), a TNFR-1 DD molecular complex, or a TNFR-1 DD analogue. The machine of the present invention comprises a machine-readable data storage medium comprising a data storage material encoded with machine-readable data. Machine-readable storage media comprising data storage material include conventional computer hard drives, floppy disks, DAT tape, CD-ROM, and other magnetic, magneto-optical, optical, floptical and other media which may be adapted for use with a computer. The machine of the present invention also comprises a working memory for storing instructions for processing the machine-readable data, as well as a central processing unit (CPU) coupled to the working memory and to the machine-readable data storage medium for the purpose of processing the machine-readable data into the desired three dimensional representation. Finally, the machine of the present invention further comprises a display connected to the CPU so that the three dimensional representation may be visualized by the user. Accordingly, when used with a machine programmed with instructions for using said data, e.g., a computer loaded with one or more programs of the sort identified above, the machine provided for herein is capable of displaying a graphical three-dimensional representation of any of the molecules or molecular complexes, or portions of molecules of molecular complexes, described herein.

In one embodiment of the invention, the machine-readable data comprises the relative structural coordinates of amino acid residues K343, E344, R347, R348 and D353, ± a root mean square deviation from the conserved backbone atoms of said amino acids of not more than 1.5 Å, or preferably, not more than 1.0 Å, or more preferably not more than 0.5 Å. In an alternate embodiment, the machine-readable data further comprises the relative structural coordinates of amino acid residues E369 and Y373 according to FIG. 8, ± a root mean square deviation from the conserved backbone atoms of said amino acids of not more than 1.5 Å, preferably not more than 1.0 Å, and most preferably not more than 0.5 Å. In another embodiment, the machine-readable data still further comprises the relative structural coordinates of amino acid residues E335, E386, E390, D398, E406, D407, E409 and E410 according to FIG. 8, ± a root mean square deviation from the conserved backbone atoms of said amino acids of not more than 1.5 Å, or preferably, not more than 1.0 Å, or more preferably not more than 0.5 Å. In yet another embodiment, the machine readable data comprises the relative structural coordinates of amino acid residues R358, R365, R368, R379, R380, R381, R384, R385, R394 and R397 according to FIG. 8, ± a root mean square deviation from the conserved backbone atoms of said amino acids of not more than 1.5 Å, or preferably, not more than 1.0 Å, or more preferably not more than 0.5 Å. Finally, in the most preferred embodiment, the machine readable data comprises the complete structural coordinates according to FIG. 8, ± a root mean square deviation of not more than 1.5 A (or more preferably, not more than 1.0 Å, and most preferably, not more than 0.5 Å).

The structural coordinates of the present invention permit the use of various molecular design and analysis techniques in order to (i) solve the three dimensional structures of related molecules, molecular complexes or TNFR-1 DD analogues, and (ii) to design, select, and synthesize chemical agents capable of favorably associating or interacting with an active site of an TNFR-1 DD molecule, molecular complex or TNFR-1 DD analogue, wherein said chemical agents potentially act as inhibitors of TNFR-1 DD or TNFR-1 DD complex binding to a number of binding proteins, including, but not limited to, TRADD DD.

More specifically, the present invention provides a method for determining the molecular structure of a molecule or molecular complex whose structure is unknown, comprising the steps of obtaining a solution of the molecule or molecular complex whose structure is unknown, and then generating NMR data from the solution of the molecule or molecular complex. The NMR data from the molecule or molecular complex whose structure is unknown is then compared to the solution structure data obtained from the TNFR-1 DD solutions of the present invention. Then, 2D, 3D and 4D isotope filtering, editing and triple resonance NMR techniques are used to conform the three dimensional structure determined from the TNFR-1 DD solution of the present invention to the NMR data from the solution molecule or molecular complex. Alternatively, molecular replacement may be used to conform the TNFR-1 DD solution structure of the present invention to x-ray diffraction data from crystals of the unknown molecule or molecular complex.

Molecular replacement uses a molecule having a known structure as a starting point to model the structure of an unknown crystalline sample. This technique is based on the principle that two molecules which have similar structures, orientations and positions will diffract x-rays similarly. A corresponding approach to molecular replacement is applicable to modeling an unknown solution structure using NMR technology. The NMR spectra and resulting analysis of the NMR data for two similar structures will be essentially identical for regions of the proteins that are structurally conserved, where the NMR analysis consists of obtaining the NMR resonance assignments and the structural constraint assignments, which may contain hydrogen bond, distance, dihedral angle, coupling constant, chemical shift and dipolar coupling constant constraints. The observed differences in the NMR spectra of the two structures will highlight the differences between the two structures and identify the corresponding differences in the structural constraints. The structure determination process for the unknown structure is then based on modifying the NMR constraints from the known structure to be consistent with the observed spectral differences between the NMR spectra.

Accordingly, in one non-limiting embodiment of the invention, the resonance assignments for the TNFR-1 DD solution provide the starting point for resonance assignments of TNFR-1 DD in a new TNFR-1 DD:“unsolved agent” complex. Chemical shift perturbances in two dimensional ¹⁵N/¹H spectra can be observed and compared between the TNFR-1 DD solution and the new TNFR-1 DD:agent complex. In this way, the affected residues may be correlated with the three dimensional structure of TNFR-1 DD as provided by the relevant structural coordinates of FIG. 8. This effectively identifies the region of the TNFR-1 DD:agent complex that has incurred a structural change relative to the native TNFR-1 DD structure. The ¹H, ¹⁵N, ¹³C and ¹³CO NMR resonance assignments corresponding to both the sequential backbone and side-chain amino acid assignments of TNFR-1 DD may then be obtained and the three dimensional structure of the new TNFR-1 DD:agent complex may be generated using standard 2D, 3D and 4D triple resonance NMR techniques and NMR assignment methodology, using the TNFR-1 DD solution structure, resonance assignments and structural constraints as a reference. Various computer fitting analyses of the new agent with the three dimensional model of TNFR-1 DD may be performed in order to generate an initial three dimensional model of the new agent complexed with TNFR-1 DD, and the resulting three dimensional model may be refined using standard experimental constraints and energy minimization techniques in order to position and orient the new agent in association with the three dimensional structure of TNFR-1 DD.

The present invention further provides that the structural coordinates of the present invention may be used with standard homology modeling techniques in order to determine the unknown three-dimensional structure of a molecule or molecular complex. Homology modeling involves constructing a model of an unknown structure using structural coordinates of one or more related protein molecules, molecular complexes or parts thereof (i.e., active sites). Homology modeling may be conducted by fitting common or homologous portions of the protein whose three dimensional structure is to be solved to the three dimensional structure of homologous structural elements in the known molecule, specifically using the relevant (i.e., homologous) structural coordinates provided by FIG. 8 herein. Homology may be determined using amino acid sequence identity, homologous secondary structure elements, and/or homologous tertiary folds. Homology modeling can include rebuilding part or all of a three dimensional structure with replacement of amino acids (or other components) by those of the related structure to be solved.

Accordingly, a three dimensional structure for the unknown molecule or molecular complex may be generated using the three dimensional structure of the TNFR-1 DD molecule of the present invention, refined using a number of techniques well known in the art, and then used in the same fashion as the structural coordinates of the present invention, for instance, in applications involving molecular replacement analysis, homology modeling, and rational drug design.

Determination of the three dimensional structure of TNFR-1 DD, its TRADD DD binding active site, and other binding sites, is critical to the rational identification and/or design of agents that may act as inhibitors of TNFR-1 DD, such as inhibitors of TRADD DD binding to TNFR-1 DD. This is advantageous over conventional drug assay techniques, in which the only way to identify such an agent is to screen thousands of test compounds until an agent having the desired inhibitory effect on a target compound is identified. Necessarily, such conventional screening methods are expensive, time consuming, and do not elucidate the method of action of the identified agent on the target compound.

However, advancing X-ray, spectroscopic and computer modeling technologies allow researchers to visualize the three dimensional structure of a targeted compound (i.e., of TNFR-1 DD). Using such a three dimensional structure, researchers identify putative binding sites and then identify or design agents to interact with these binding sites. These agents are then screened for an inhibitory effect upon the target molecule. In this manner, not only are the number of agents to be screened for the desired activity greatly reduced, but the mechanism of action on the target compound is better understood.

Accordingly, the present invention further provides a method for identifying a potential inhibitor of TNFR-1 DD, a TNFR-1 DD analogue or of a TNFR-1 DD complex, comprising the steps of using a three dimensional structure of TNFR-1 DD as defined by the relative structural coordinates of FIG. 8 to design or select a potential inhibitor of TNFR-1 DD activity, and synthesizing or obtaining said potential inhibitor. The inhibitor may be selected by screening an appropriate database, may be designed de novo by analyzing the steric configurations and charge potentials of an empty TNFR-1 DD or TNFR-1 DD complex active site in conjunction with the appropriate software programs, or may be designed using characteristics of known inhibitors of protein binding to TNFR-1 DD or TNFR-1 DD complexes in order to create “hybrid” inhibitors.

An agent that interacts or associates with an active site of TNFR-1 DD, a TNFR-1 DD complex or a TNFR-1 DD analogue may be identified by determining an active site from the three dimensional structure of TNFR-1 DD, and performing computer fitting analyses to identify an agent which interacts or associates with said active site. Computer fitting analyses utilize various computer software programs that evaluate the “fit” between the putative active site and the identified agent, by (a) generating a three dimensional model of the putative active site of a molecule or molecular complex using homology modeling or the atomic structural coordinates of the active site, and (b) determining the degree of association between the putative active site and the identified agent. The degree of association may be determined computationally by any number of commercially available software programs, or may be determined experimentally using standard binding assays.

Three dimensional models of the putative active site may be generated using any one of a number of methods known in the art, and include, but are not limited to, homology modeling as well as computer analysis of raw structural coordinate data generated using crystallographic or spectroscopy techniques. Computer programs used to generate such three dimensional models and/or perform the necessary fitting analyses include, but are not limited to: GRID (Oxford University, Oxford, UK), MCSS (Molecular Simulations, San Diego, Calif.), AUTODOCK (Scripps Research Institute, La Jolla, Calif.), DOCK (University of California, San Francisco, Calif.), Flo99 (Thistlesoft, Morris Township, N.J.), Ludi (Molecular Simulations, San Diego, Calif.), QUANTA (Molecular Simulations, San Diego, Calif.), Insight (Molecular Simulations, San Diego, Calif.), SYBYL (TRIPOS, Inc., St. Louis, Mo.) and LEAPFROG (TRIPOS, Inc., St. Louis, Mo.).

In a preferred method of the present invention, the active site preferably comprises amino acid residues K343, E344, R347, R348 and D353 (or conservative substitutions thereof) according to FIG. 6, amino acid residues E369 and Y373 (or conservative substitutions thereof) according to FIG. 6, amino acid residues E335, E386, E390, D398, E406, D407, E409 and E410 (or conservative substitutions thereof) according to FIG. 6, or amino acid residues R358, R365, R368, R379, R380, R381, R384, R385, R394 and R397 (or conservative substitutions thereof) according to FIG. 6.

In the more preferred embodiment, the method of the present invention includes the use of an active site characterized by the three dimensional structure comprising the relative structural coordinates of amino acid residues K343, E344, R347, R348 and D353 according to FIG. 8, ± a root mean square deviation from the conserved backbone atoms of said amino acids of not more than 1.5 Å, preferably not more than 1.0 Å, and most preferably not more than 0.5 Å. In another embodiment, the active site is characterized by the three dimensional structure comprising the relative structural coordinates of amino acid residues E369 and Y373 according to FIG. 8, ± a root mean square deviation from the conserved backbone atoms of said amino acids of not more than 1.5 Å, preferably not more than 1.0 Å, and most preferably not more than 0.5 Å. In another embodiment, the active site is characterized by the three dimensional structure comprising the relative structural coordinates of amino acid residues E335, E386, E390, D398, E406, D407, E409 and E410 according to FIG. 8, ± a root mean square deviation from the conserved backbone atoms of said amino acids of not more than 1.5 Å, preferably not more than 1.0 Å, and most preferably not more than 0.5 Å. In yet another embodiment, the active site is characterized by the three dimensional structure comprising the relative structural coordinates of amino acid residues R358, R365, R368, R379, R380, R381, R384, R385, R394 and R397 according to FIG. 8, ± a root mean square deviation from the conserved backbone atoms of said amino acids of not more than 1.5 Å, preferably not more than 1.0 Å, and most preferably not more than 0.5 Å. It is understood that the method of the present invention includes additional embodiments comprising conservative substitutions of the noted amino acids which result in the same structural coordinates of the corresponding residues in FIG. 8 within the stated root mean square deviation.

The effect of such an agent identified by computer fitting analyses on TNFR-1 DD, TNFR-1 DD complex or TNFR-1 DD analogue activity may be further evaluated computationally, or experimentally by competitive binding experiments or by contacting the identified agent with TNFR-1 DD (or a TNFR-1 DD complex or analogue or a molecule comprising TNFR-1 DD such as TNFR-1) and measuring the effect of the agent on the target's biological activity. Standard enzymatic assays may be performed and the results analyzed to determine whether the agent is an inhibitor of TNFR-1 DD activity (i.e., the agent may reduce or prevent binding affinity between TNFR-1 DD and a relevant binding protein, such as TRADD DD. Further tests may be performed to evaluate the selectivity of the identified agent to TNFR-1 DD with regard to other TNFR-1 DD analogues or TRADD DD binding targets.

An agent designed or selected to interact with TNFR-1 DD preferably is capable of both physically and structurally associating with TNFR-1 DD via various covalent and/or non-covalent molecular interactions, and of assuming a three dimensional configuration and orientation that complements the relevant active site of TNFR-1 DD.

Accordingly, using these criteria, the structural coordinates of the TNFR-1 DD molecule as disclosed herein, and/or structural coordinates derived therefrom using molecular replacement or homology modeling, agents may be designed to increase either or both of the potency and selectivity of known inhibitors, either by modifying the structure of known inhibitors or by designing new agents de novo via computational inspection of the three dimensional configuration and electrostatic potential of a TNFR-1 DD active site.

Accordingly, in one embodiment of the invention, the structural coordinates of FIG. 8 of the present invention, or structural coordinates derived therefrom using molecular replacement or homology modeling techniques as discussed above, are used to screen a database for agents that may act as potential inhibitors of TNFR-1 DD activity. Specifically, the obtained structural coordinates of the present invention are read into a software package and the three dimensional structure is analyzed graphically. A number of computational software packages may be used for the analysis of structural coordinates, including, but not limited to, Sybyl (Tripos Associates), QUANTA and XPLOR (Brunger, A. T., (1994) X-Plor 3.851: a system for X-ray Crystallograph and NMR. Xplor Version 3.851 New Haven, Conn.: Yale University Press). Additional software programs check for the correctness of the coordinates with regard to features such as bond and atom types. If necessary, the three dimensional structure is modified and then energy minimized using the appropriate software until all of the structural parameters are at their equilibrium/optimal values. The energy minimized structure is superimposed against the original structure to make sure there are no significant deviations between the original and the energy minimized coordinates.

The energy minimized coordinates of TNFR-1 DD bound to a “solved” inhibitor are then analyzed and the interactions between the solved ligand and TNFR-1 DD are identified. The final TNFR-1 DD structure is modified by graphically removing the solved inhibitor so that only TNFR-1 DD and a few residues of the solved agent are left for analysis of the binding site cavity. QSAR and SAR analysis and/or conformational analysis may be carried out to determine how other inhibitors compare to the solved inhibitor. The solved agent may be docked into the uncomplexed structure's binding site to be used as a template for data base searching, using software to create excluded volume and distance restrained queries for the searches. Structures qualifying as hits are then screened for activity using standard assays and other methods known in the art.

Further, once the specific interaction is determined between the solved inhibitor, docking studies with different inhibitors allow for the generation of initial models of new inhibitors bound to TNFR-1 DD. The integrity of these new models may be evaluated a number of ways, including constrained conformational analysis using molecular dynamics methods (i.e., where both TNFR-1 DD and the bound inhibitor are allowed to sample different three dimensional conformational states until the most favorable state is reached or found to exist between the protein and the bound agent). The final structure as proposed by the molecular dynamics analysis is analyzed visually to make sure that the model is in accord with known experimental SAR based on measured binding affinities. Once models are obtained of the original solved agent bound to TNFR-1 DD and computer models of other molecules bound to TNFR-1 DD, strategies are determined for designing modifications into the inhibitors to improve their activity and/or enhance their selectivity.

Once a TNFR-1 DD binding agent has been optimally selected or designed, as described above, substitutions may then be made in some of its atoms or side groups in order to improve or modify its selectivity and binding properties. Generally, initial substitutions are conservative, i.e., the replacement group will have approximately the same size, shape, hydrophobicity and charge as the original group. Such substituted chemical compounds may then be analyzed for efficiency of fit TNFR-1 DD by the same computer methods described in detail above.

Various molecular analysis and rational drug design techniques are further disclosed in U.S. Pat. Nos. 5,834,228, 5,939,528 and 5,865,116, as well as in PCT Application No. PCT/US98/16879, published as WO 99/09148, the contents of which are hereby incorporated by reference.

The present invention may be better understood by reference to the following non-limiting Examples. The following Examples are presented in order to more fully illustrate the preferred embodiments of the invention, and should in no way be construed as limiting the scope of the present invention.

### EXAMPLE 1

Tumor Necrosis Factor Receptor 1 (TNFR-1) death domain (DD) is the intracellular functional domain responsible for the receptor signaling activities. To understand the transduction mechanism of TNFR-1 signaling, we have performed structural and functional analysis of the TNFR-DD. The secondary structure of the TNFR-DD shows that it consists of six antiparallel helices. The determination of the topological fold and an extensive mutagenesis analysis revealed that there are two opposite faces that are involved in self-association and interaction with the TRADD death domain. Interestingly, the same critical residues in TNFR-DD are involved in both these interactions. There is a good correlation between the binding activities of the mutant proteins and their cytotoxic activities. These results provide important insight into the molecular interactions mediating TNFR-DD self-association and subsequent recruitment of TRADD in the signaling activity of TNFR-1.

Experimental Methods

TNFR-DD cloning and site-directed mutagenesis. The DNA sequence coding from amino acid A316 to R426 from hTNFR-1 was cloned in pRSETB (Invitrogen) after amplification by PCR. The primer at the 5′ end introduces an initiation site with an NdeI site upstream the methionine, and the primer at the 3′ end introduces a His tag after R426. The sequence was confirmed by sequencing. Single point mutations were introduced using the chameleon double-stranded site directed mutagenesis (Stratagene) and verified by sequencing of the coding region.

Preparation of R347A mutant for NMR studies. 20 grams of wet cell paste expressing TNFR-DD R347A were resuspended in 300 ml lysis buffer containing 50 mM Tris-Cl, pH 8.8, 50 mM NaCl, and protease inhibitor tablets (Boehringer Mannheim GmbH, Mannheim, Germany). Cells were lysed by two passages through a Microfluidizer (Microfluidics Corporation, Newton, Mass.). Cellular debris were removed by centrifugation at 15000 g for 30 min. The soluble extract was then loaded onto a 2×20 cm Ni-NTA Superflow (Qiagen) column pre-equilibrated with lysis buffer. The column was washed with large amount of lysis buffer until no protein was present in the flow-through as judged by Bradford protein assay. TNFR-DD R347A was eluted with a step elution of 400 mM Imidazole in lysis buffer. DTT was added immediately to the eluate to a final concentration of 10 mM. Death domain containing fractions were pooled and dialysed against buffer A (50 mM Tris.Cl, pH 8.8, 50 Imidazole, 10 mM DTT) overnight, and applied onto a 1×10 cm Toyopearl QAE-550C anion exchange column (TosoHaas, Montgomeryville, Pa.) pre-equilibrated with buffer A. Greater than 95% pure R347A mutant was eluted with a linear NaCl gradient. The fractions were subjected to SDS-PAGE analysis and good fractions were combined and concentrated before loading onto a Superdex 75 gel filtration column (Amersham Pharmacia Biotech, Piscataway, N.J.) equilibrated with buffer containing 50 mM Tris.Cl, pH 8.8, 100 mM NaCl, 50 mM Imidazole, 10 mM DTT. R347A containing fractions were eluted, dialysed and concentrated for NMR studies.

NMR spectroscopy. All NMR spectra were acquired at 25° C on Varian Unity+600 spectrometer. A series of HSQC [24] related three dimensional triple-resonance experiments were recorded using uniformly ¹⁵N- and ¹⁵N/¹³C-labeled protein in water with 5% D₂O, including HNCO, (HB)CBCACONNH, HNCACB, C(CC)_TOCSY_(CO)NNH, H(CC)_TOCSY_(CO)NNH, and HAHB(CBCA)CONNH, and HN(CACB)HAHB [23,24] to make backbone, side-chain and sequential assignments. 3D-HNHA [38] and 2D HMQC-J [50] were carried out for ³J_{NHα} coupling constant measurement. R347A is a proline rich protein that contains a total of nine proline residues. The HBCBCA(CO)_N_CAHA and HACA_N [28], which correlates sequential residues via their ¹⁵N nuclei were used to assign proline residues and additional amide residues which were missing due to fast exchange with water at pH 8.8. Validation and additional residue assignments were made using both ¹⁵N-edited NOESY and ¹³C-edited NOESY [29, 27] experiments. Data sets were processed and displayed on SGI work station using the programs NMRDraw and NMRPipe [30]. The programs PIPP and STAPP [32] were used for peak picking, data analysis and part of auto-sequential assignment.

TNFR-DD three-dimensional structural model. The TNFR-DD sequence alignment with Fas-DD was determined using QUANTA98 (Molecular Simulations, Inc., San Diego, Calif.). An initial homology model for TNFR-DD based on its sequence alignment with Fas-DD was accomplished with MODELLER [54, 55]. The TNFR-DD homology model was further defined to maintain consistency with the observed secondary structure elements observed by NMR. Basically, the structure was subjected to a simulated annealing protocol (40) using the program XPLOR (42) where only regions of the protein that differed from the secondary structure observed by NMR were free to move. φ and ψ dihedral constraints based on accepted values for standard secondary structure elements were applied to residues identified to be α-helical by NMR, otherwise no constraints were applied and the residue was free to move. The Fas-DD sequence and solution structure (PDB accession code 1DDF) were used as reported by Huang, et al. [12].

Protein preparation for binding assay. Wild-type and mutant recombinant TNFR-DD proteins were prepared from Escherichia Coli strain BL21 (DE3) pLysS (Stratagene). When cell growth reached an OD₅₉₅ of ˜0.5 at room temperature, protein expression was induced by adding 0.3-mM isopropyl-1-thio-β-D-galactopyranoside for 4 h. Cells were lysed with the french press (1000 psi) in 50 mM Tris pH 7.5, 50 mM NaCl, 10 μg/ml leupeptin, 10 μg/ml aprotinin, 2 mM phenylmethylsulfonyl fluoride at 4° C. The lysate was centrifuged at 21000 g at 4° C. for 30 minutes. The soluble mutants were then incubated for 1 hour with Ni-NTA resin (Qiagen) at 4° C. The resin was washed twice in 50 mM Tris pH 7.5, 50 mM NaCl and twice in 50 mM Tris pH 7.5, 50 mM NaCl, 50 mM imidazole. The bound protein was eluted from the resin in 50 mM Tris pH 7.5, 50 mM NaCl, 250 mM imidazole and 10 mM DTT was added to the eluate. The wild-type protein and most of the mutants were in the pellet fraction after lysis. The pellet was then washed twice in 50 mM Tris pH 8.5, 50 mM NaCl and the death domain proteins were extracted in 50 mM NaOAc pH 4.0, 50 mM MgSO4. 10 mM DTT was then added to the extract.

Effect of ionic strength on TNFR-DD self-association and association with TRADD-DD. The experiments were carried out according to the protocol described in the next paragraph (TNFR-DD binding assay). Only, the binding buffer was supplemented with NaCl 5M to obtain the desired final concentrations of 140 mM, 300 mM, 600 mM and 1.2 M.

TNFR-DD binding assay. TNFR-DD binding to itself or to TRADD was determined by an enzyme-linked immunosorbent assay (ELISA). Ninety six well plates were coated overnight at 4° C. with 200 ng of MBP-TNFR-DD, MBP-TRADD or MBP-MADD-LZ (N-terminal region of MADD containing a leucine zipper motif) in coating buffer (25 mM MOPS pH 7.5, 150 mM NaCl, 10 mM DTT, 0.02% NaN₃). The plates were then incubated in blocking buffer (10 mM MOPS 7.5, 150 mM NaCl, 0.05% Tween 20, 0.1% Gelatin, 0.02% NaN₃) for one hour followed by a one hour incubation with the different TNFR-DD proteins in binding buffer (100 mM Tris 7.5, 140 mM NaCl, 0.1 mM EDTA, 0.2% Triton X-100, 10 mM DTT). The plates were washed 4 times in 10 mM KP04 pH 7.4, 0.05% Tween 20. The bound His tagged proteins were then incubated for 30 minutes in 10 mM MOPS 7.5, 150 mM NaCl, 0.05% Tween 20, 0.02% NaN₃, anti-6x His mAb (1:1000), anti-mouse Biotin (1:1000), Streptavidin-Alkaline Phosphatase (1:1000). The plates were washed 4 times in 10 mM KP04 pH 7.4, 0.05% Tween 20. Tropix CDP-Star/Sapphire II solution was used for the luminescent reaction and detection with the lumicount plate reader (Packard).

Results

Structure determination. Structure determination of the wild-type death domain of TNFR-1 by NMR has been hindered due to low protein solubility over a wide range of pH (4-10). Therefore, a series of single point mutants have been made and tested for their solubility at different pH. The mutant R347A was found to be soluble and stable in concentrations up to 14 mg/ml (˜1 mM) at pH 8.8. Under these conditions, the protein was shown to be predominantly monomeric by size exclusion chromatography. The HSQC spectrum of ¹⁵N-labeled protein indicates that the protein was folded. Therefore, this mutant protein was chosen for the subsequent NMR studies.

However, many of the amide proton peaks were severely broadened due to rapid exchange with water at high pH (pH 8.8) which hindered the complete assignment of TNFR-DD by HSQC-related triple resonance experiments. Nevertheless, the majority of TNFR-DD forms α-helices, where the amide proton exchange is reduced by hydrogen bond formation, allowing for the observation of backbone chemical shifts and the determination of sequential assignments in the helical regions. A total of 63 residues were assigned in the region from A328 to C413, where the amide signals of terminal residues, A316-P327 and G414-R426 were not be observed. In an effort to complete the sequential assignment, two experiments were designed based on recently developed methodologies where NMR experiments correlated sequential residues via ¹⁵N and Hα chemical shifts instead of the amide proton. These experiments enabled us to assign 24 additional residues. Therefore, a total of 87 residues out of 112 amino acids were assigned.

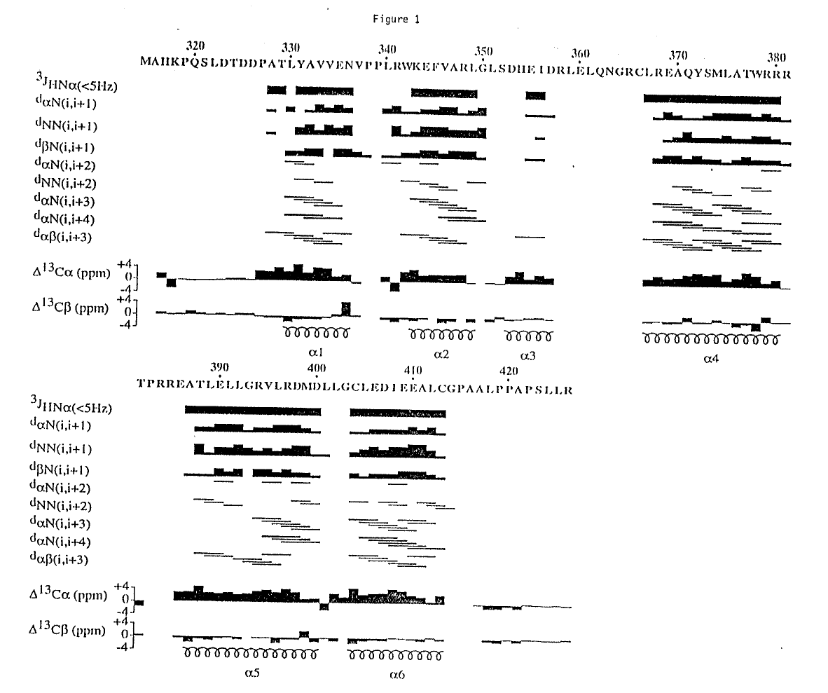

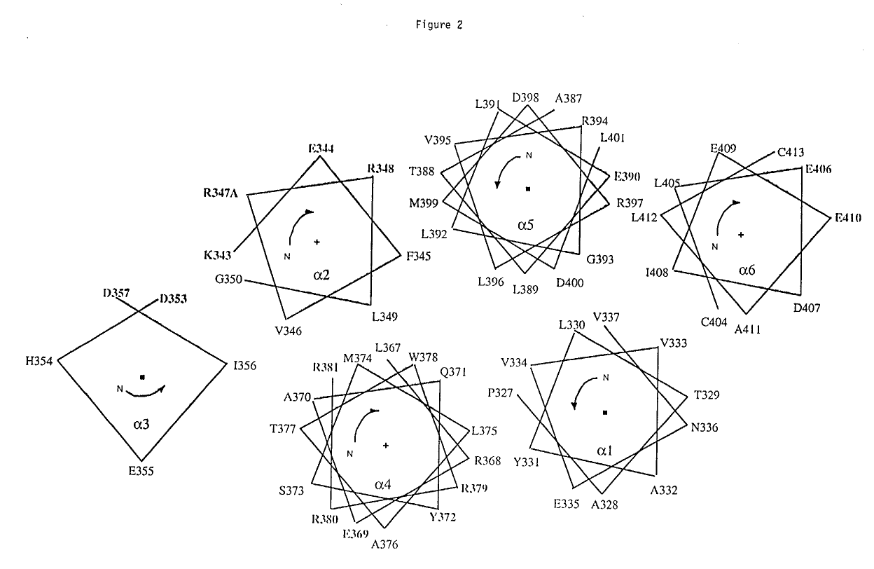

FIG. 1 summarizes the NMR secondary structure data for TNFR-DD R347A. This data indicates that TNFR-DD consists of six α-helices where helices 1, 2, 4, 5 and 6 exhibit the typical NOE pattern for an helix (³JHN_{α}<5Hz, short- and medium-range NOEs: d_{NN}(i,i+1), d_{αN}(i,i+3) d_{60 N}(i,i+4) and d_{αβ}(i,i+3) and Cα/Cβ secondary chemical shifts). These helices are separated by loop regions that were not observed because of the lack of amide proton resonance peaks due to the rapid solvent exchange. Cα/Cβ chemical shift deviation indicates that both terminal regions are unstructured. A comparison of the secondary structure of TNFR-DD with Fas-DD indicates that the main structural difference between the two proteins resides in the length of helix-3. Helix 3 in the Fas-DD structure encompass nine residues from D240 to E248, whereas in TNFR-DD R347A, only three amide protons (E355 to D357) were detectable. The lack of amide peaks in the C-terminus region hinders the assignment of the secondary structure for these residues. However, the relatively high amide exchange rate suggests that the structure of this region is somewhat disordered, possibly due to a higher flexibility of helix-3. Data obtained by D₂O exchange with the mutant R347K at pH4 supports this observation (data not shown). Interestingly, similar results were obtained with FADD-DD [13]. The two dimensional helical wheel topology shown in FIG. 2 is based on more than 40 long range inter-helical side chain NOEs assigned from ¹³C edited NOESY experiments, combined with secondary structure analysis (FIG. 1). TNFR-DD is consistent with other published DD structures where the protein consists of six helices arranged in an anti-parallel fashion. Many of the long range NOEs were found between side chain residues of α2 making contacts with side chain residues of α3 (K343-I356, V346-I356, A347-I356), α4 (V346-M374, L349-M374, L349-W378) and α5 (F345-L389, L349-L392, L349-V395, R348-V395). A few NOE pairs involving contacts between α1 and α4 (V334-Q371) as well as between α1 and α6 (V333-I408 and V337-I408) were also found. Only one side chain contact between α5 and α6 was observed, with L401 at the C-terminus of α5 and L405 at the N-terminus of α6, suggesting that the angle between these two helices is such that it prevents additional side chain contacts between them. Based on the planar view centered on helix-2 (FIG. 2), helices 2-5 form a twisted four helix bundle with a hydrophobic core consisting of residues F345, V346, I356, M374, L392. The side chain of the hydrophilic residues in the helical bundle are solvent exposed. The long range inter-helical NOE between side chain of the residues in the core region indicates that these residues are close to each other and probably form hydrophobic interactions to stabilize the protein folding.

The secondary structure assignment of R347A is very similar to that of the mutant R347K, which has been shown to have a similar structure to that of the wild-type protein, based on HSQC spectra comparison performed at pH4.0 (data not shown). Therefore, the overall structure of R347A is most likely similar to that of the wild-type.

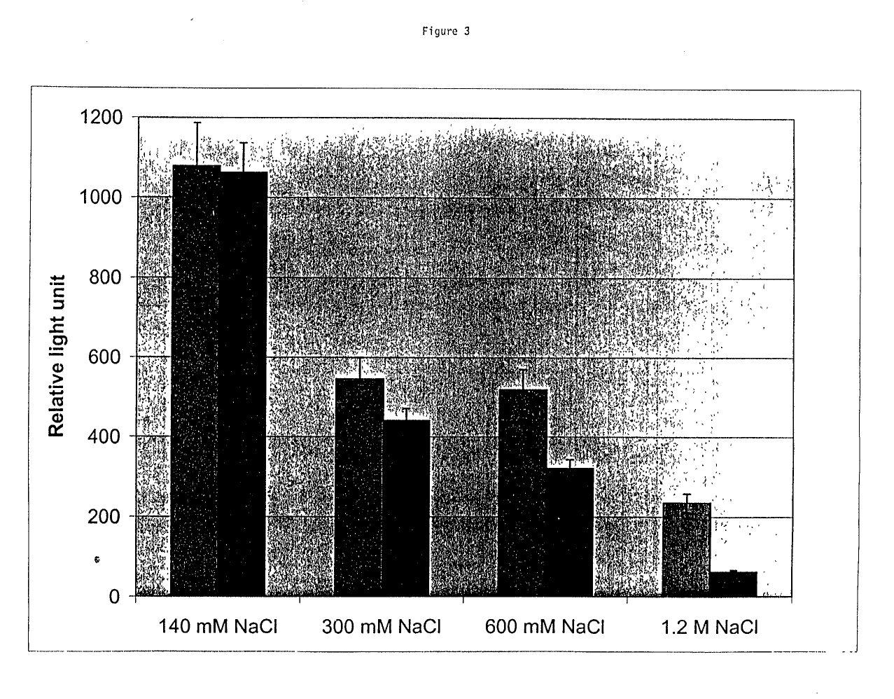

Effect of ionic strength on TNFR-DD self-association and its interaction with TRADD-DD. Based on the observation that charged residues have been shown to be involved in the Fas-FADD death domains interaction, we examined the effect of sodium chloride on the TNFR-DD self association and the interaction with TRADD death domains. FIG. 3 shows that increasing concentrations of NaCl are accompanied by a decrease in death domain interaction in both cases, implying that electrostatic interactions and/or hydrogen bonds are important for the TNFR-death domain self-association and its interaction with TRADD-DD.

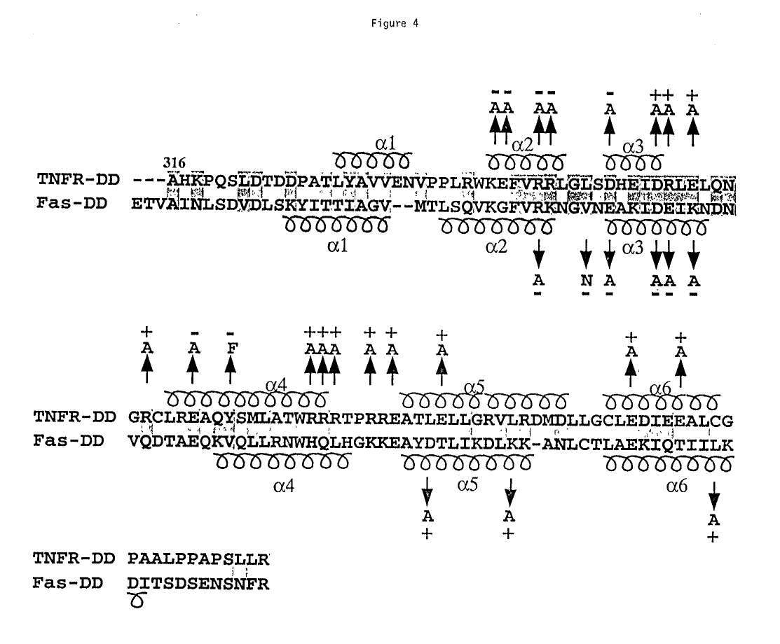

Effect of TNFR-DD single point mutations on self-association. Since electrostatic interactions might play an important role in death domain interactions, a series of 19 charged amino acids were mutagenized (FIG. 4). Alanine was chosen as the substituent amino acid, since it eliminates the contribution from the side chain of the replaced amino acid with a potential minimal alteration of the overall protein structure. For tyrosine 372, the substitution was made with a phenylalanine in order to abolish the contribution of the hydroxyl group while keeping the aromatic side chain intact. The wild-type and mutant constructs were expressed in bacteria as His-tagged proteins at the C-terminus. The wild-type and most of the mutant proteins were in the insoluble fraction after lysis at pH7.5. Only mutants E344A and R347A, were soluble upon lysis at pH7.5. These mutant proteins were purified from the lysate with Ni-NTA resin. All the others including the wild-type were extracted directly from the pellet fraction at pH 4.0. The purity of the proteins was determined to be about 80% by SDS-PAGE.

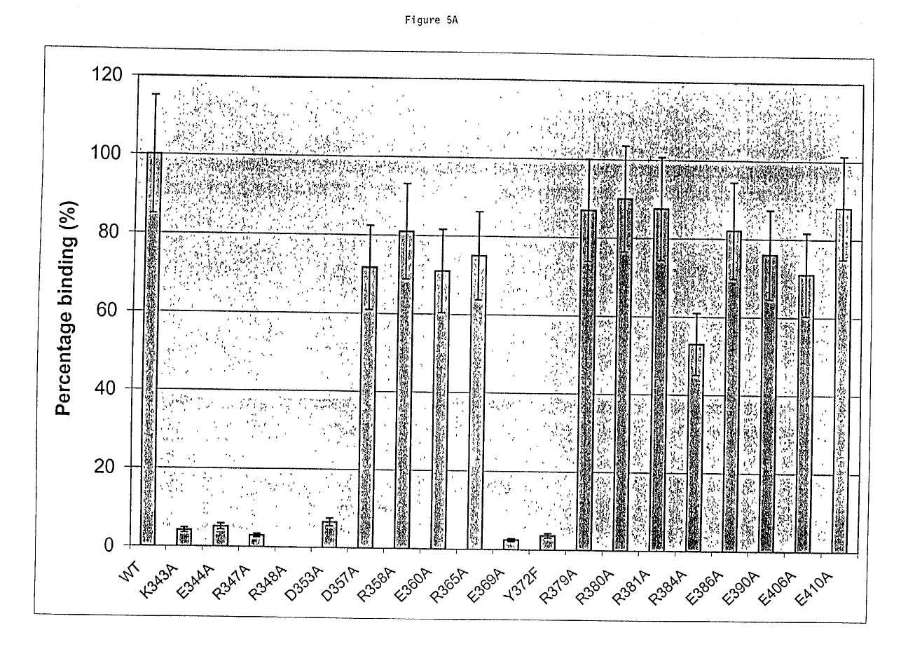

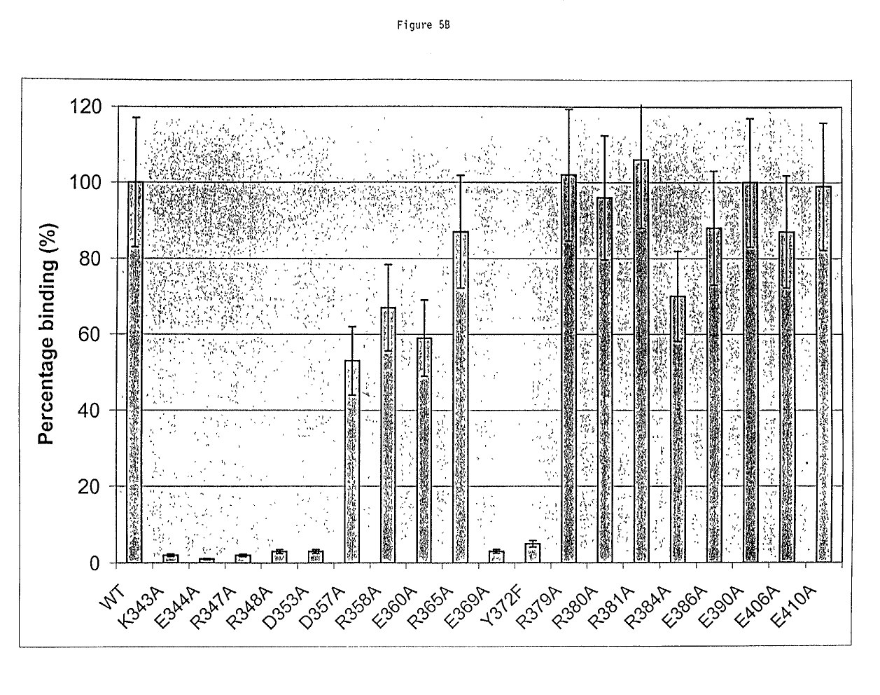

All the mutants depicted in FIG. 4 were studied for their ability to interact with the wild-type death domain in a cell free binding assay. The wild-type TNFR-DD was expressed as a MBP fusion protein (MBP-TNFR-DD) and was immobilized on 96 well plates for ELISA. Despite the tendency of the wild type and most of the mutant proteins to aggregate at pH 7.5, all of them were soluble at that pH over the concentration range that was used for these binding experiments (0.1-5 μg/ml). The four mutations K343A, E344A, R347A and R348A in α2 abolish the interaction with the wild-type death domain (FIG. 5A). Mutation of the first residue of α3, D353A, also abolishes the interaction with the wild-type death domain. Conversely, the next three sequential mutations in α3, D357A, R358A and E360A, and R365A in loop 3 retain an interaction with MBP-TNFR-DD similar to that of the wild-type protein. The first two sequential mutations in α4, E369A and Y372F, lost their interaction with the wild-type death domain similarly to the mutants in α2. All the other mutations in α4 (R379A, R380A, R381A), Loop4 (R384A, E386A), α5 (E390A) and α6 (E406A, E410A) retained an interaction with MBP-TNFR-DD similar to that of the wild-type death domain. Effect of TNFR-DD single point mutations on interaction with TRADD-DD. The TNFR-DD mutants were also studied for their ability to interact with TRADD. The full length TRADD protein was expressed as a MBP fusion protein (MBP-TRADD) and was immobilized on 96 well plates for ELISA. The trend of the binding ability of all the different mutants to MBP-TRADD is the same as for the binding to MBP-TNFR-DD (FIG. 5B).

Discussion

We have performed NMR studies and extensive mutagenesis analysis of the TNFR-DD. Overall, the secondary structure of TNFR-DD is similar to the structure of other death domains. It consists of six antiparallel α-helices. Interestingly, it appears that α2, part of α3 and part of α4 contain important residues for the self-association and interaction with TRADD-DD. Individual mutation of the four charged residues in α2, K343A, E344A, R347A and R348A results in a loss of interaction with TNFR-DD and with TRADD-DD. These residues are solvent exposed and located on the same side of the protein according to the deduced topological fold (FIG. 2). On the other hand, only the first mutated residue in α3, D353A, has the same effect. The other three mutations in α3 (D357A, R358A and E360A) have no or little effect on the binding properties of the TNFR-DD. In the case of α4, the first two mutants E369A and Y372F are deficient in their interaction with TNFR-death domain and TRADD-DD. The other three mutations, R379A, R380A, R381A retain binding properties that are similar to the wild-type protein. The other mutants in loop 3 (R365A), loop 4 (R384A and E386A), α5 (E390A) and α6 (E406A and E410A) also seem to be unaffected in their binding properties when compared to the wild-type protein. These results are in good agreement with the cytotoxicity data obtained previously by [7, 10]. Mutants R347A and E369A that are defective in our ELISA binding assays are also deficient in cytotoxicity. In contrast, mutants R379A, E390A, E406A and E410A that have unaltered binding properties also retained cytotoxic activity. Only mutant K343A that lost its ability to interact with the wild-type receptor DD or with TRADD-DD retained some cytotoxic activity, also to a lesser extent than the wild-type receptor [7, 10]. The correlation between death domain interaction and cytotoxicity further supports the hypothesis that the aggregation of TNFR-1 and subsequent recruitment of TRADD through death domain interactions is critical for generating a cytotoxic signal [8, 51, 52]. From our results, it is possible that self-association and binding to TRADD-DD use similar surfaces since all the mutations have the same effect in both cases. When these mutations are placed in the context of the topological fold, it appears that there are two opposite and solvent exposed faces involved in the interaction. On one side there are critical residues in α2 (K343A, E344A, R347A and R348A) and the beginning of α3 (D353A) and on the other side there are critical residues in α4 (E369A, Y372F). It was recently shown that α2 and α3 of FADD-DD contain critical residues for the interaction with Fas-DD [13]. However, the role of α4 in FADD-DD has yet to be addressed.

Interestingly, a comparison of mutations in α3 of Fas and TNFR-1 death domains reveals differences in the contribution of this helix to their respective binding properties. Regarding the self-association, the four mutations in α3 of Fas (E240A, D244A, E245A and K247A) abrogate their self-association [12]. On the contrary, in the case of TNFR-1, for the four equivalent mutations, only the first one in α3 (D353A) abrogates the interaction with the wild type death domain, as the next three mutations (D357A, R358A and E360A) do not (FIG. 4). Therefore, it would seem that the entire helix-3 of Fas-DD is important for the self-association, as in the case of TNFR-DD only the beginning of helix-3 is important for the self-association. In the case of the Fas-FADD interaction, the first two mutations in α3 of Fas-DD, E240A and D244A greatly affect their interaction with FADD-DD, as the next two mutations E245A and K247A only have a marginal effect. In the case of TNFR-1-TRADD interaction, only the first mutation in α3 of TNFR-DD (D353A) abrogates the interaction with TRADD-DD, as the next three mutations (D357A, R358A and E360A) do not. It is worth mentioning that while self-association of Fas-DD may not be required for the recruitment of FADD to the receptor, as suggested by the mutants E245A and K247A, we were unable to find mutations in TNFR-DD that have a differential effect on self-association and TRADD binding. Thus, it appears that α3 contains critical residues that can account for the differences between TNFR-1 and Fas regarding their self-association and interaction with other death domain proteins.

The difference between death domain interaction pairs is further demonstrated by the complex of the death domains of Pelle and Tube. The three dimensional structure of a complex between the death domains of Pelle and Tube was recently published [19]. It shows that the surfaces involved in the interaction seem to be different between different pairs of death domains. The Tube-Pelle dimers rely on contacts between α4 and α5 of Pelle with α6 and a C-terminal tail of Tube. This is in contrast with the homo- and hetero-association of the death domains of TNFR-1 and TRADD, which mainly rely on L12, (3 and α4 of TNFR-1. These results suggest that while a topological fold is maintained between members of the death domain family, the observed selectivity and specificity is obtain by a combination of distinct sequences and surface interpositions between the death domains.

Because of the charged nature of the death domains and the caspase recruitment domains, it was previously proposed that electrostatic interactions might mediate the homotypic interaction [12, 13]. However, the crystal structure of the CARD of Apaf-1 in complex with the CARD of procaspase-9 has been solved, showing that the specificity as well as the driving force for this interaction is provided by a combination of extensive hydrogen bond contacts and van der Waals interactions [18]. Moreover, this interaction is insensitive to high ionic strength, confirming that the driving force of the interaction is not mediated by electrostatic interactions. Similarly in the case of Pelle and Tube death domains interaction, it has been shown that hydrogen bonds and van der Waals interactions are the driving force for this interaction [19]. In the case of TNFR-DD, we have shown that interaction with the wild-type death domain and interaction with TRADD-DD is disrupted at high salt concentration (0.3 M-1.2M). These results suggest that TNFR-DD interactions are mediated, at least in part, by electrostatic interactions, similarly to what has been shown in the case of Fas-DD [12]. This is also supported by our mutagenesis study in which mutation of many of the charged residues to alanine induces a loss of self interaction or interaction with TRADD-DD. These different studies show that not only the surfaces, but also the nature of the interactions involved in death domain associations is not conserved, which provides for a mechanism of death domain selectivity and specificity of interaction.

A homology model for TNFR-DD was built utilizing the Fas-DD three-dimensional structure [12]. This model of TNFR-DD as well as the two dimensional helical wheel topology (FIG. 2) show that there are two opposite faces involved in TNFR-DD self-association and interaction with TRADD-DD. In TNFR-1 signaling, the receptor trimerizes upon binding with a TNF trimer and then recruits TRADD. Our experiments show that the critical residues for TNFR-DD self-association or interaction with TRADD-DD are the same. Therefore, it is possible that self-association of TNFR-DD and interaction with TRADD-DD occurs via the same binding surface. If this is the case, it is possible that ligand-induced trimerization of the receptor recruits TRADD by simply increasing the affinity for TRADD due to an effective increase in the local concentration of TNFR-DD. Alternatively, TNFR-1 trimerization may create a high affinity-binding site for TRADD. It is also possible that a third partner is involved in the regulation of the recruitment of TRADD to TNFR-1; Recently, SODD was shown to be constitutively bound to TNFR-1 intracellular domain and released from the receptor upon binding of the ligand, allowing for TRADD to be recruited to TNFR-1 [53]. It is possible that SODD prevents TRADD from interacting with TNFR-1 in the absence of ligand bound to the receptor.

In conclusion, TNFR-DD consists of six antiparallel helices similar to other death domains where the major structural and functional difference occurs in α3. Based on mutagenesis study, helices oC2 and part of α3 constitute a binding surface on one side of the protein, while α4 constitutes another binding surface on the opposite side of the protein. The critical residues involved in self-association as well as interaction with TRADD-DD are the same, suggesting a very similar mode of interaction in both cases.

### EXAMPLE 2

The solution structure of the R347K mutant of the TNFR-1 DD was solved by NMR spectroscopy. A total of 20 structures were calculated by means of hybrid distance geometry-simulated annealing using a total of 1360 experimental NMR restraints. The atomic rms distribution about the mean coordinate positions for the 20 structures for residues composing the six α-helices is 0.40 A for the backbone atoms and 1.09 A for all atoms. The structure consists of six anti-parallel α-helices arranged in a similar fashion to the other members of the death domain superfamily. The secondary structure of R347K TNFR1-DD is very similar to the secondary structure deduced for the R347A TNFR1-DD mutant (Example 1) with the exception of the length of helix 3. Helix 3 was observed to be shorter by three residues in the R347A TNFR1-DD mutant presumably a result of the inherent flexibility of this helix and the increase amide exchange rate at pH 8.8. The 3-dimensional structure of R347K TNFR-1 DD is consistent with the deduced topology of R347A TNFR-1 DD. Mutagenesis studies set forth in Example 1 identified critical residues located in helix 2 and part of helices 3 and 4 that are crucial for self-interaction and interaction with the TRADD DD. Structural superposition with previously solved proteins in the death domain superfamily reveals that the major differences between the structures reside in helices 2, 3, and 4.

Experimental Methods

TNFR-DD cloning and site-directed mutagenesis (R347K TNFR-1 DD). The DNA sequence coding from amino acid 316 to 426 from hTNFR-1 was cloned in pRSETB (Invitrogen) after amplification by PCR. The primer at the 5′end introduces an initiation site with an NdeI site upstream of the methionine, and the primer at the 3′end introduces a His tag after amino acid 426. The sequence was confirmed by sequencing analysis. A single point mutation (R347K) were introduced using the chameleon double-stranded site directed mutagenesis (Stratagene) and verified by sequencing of the coding region.

Protein expression. R347K TNFR-1 DD protein was overexpressed in BL-21 E. coli. Cells were grown at 25° C. on minimal medium containing either 2g/L ¹⁵N ammonium sulfate or ¹⁵N ammonium sulfate and 2 g/L ¹³C glucose. The cells were then lysed and R347K TNFR-1 DD mutant protein was purified (see purification section).

Purification of R347K TNFR-1 DD protein. 20 grams of wet cell paste expressing R347K TNFR-1 DD were resuspended in 300 ml lysis buffer containing 50 mM Tris-Cl, pH 7.5, 50 mM NaCl, and protease inhibitor tablets (Boehringer Mnnheim GmbH, Mannheim, Germany). Cells were lysed by two passages through a Microfluidizer (Microfluidics Corporation, Newton, Mass.). The cell lysate was centrifuged at 15000 g for 30 min. The TNFR-1 DD protein was then extracted from the pellet with 200 mL pH 8.8 buffer (50 mM Tris pH 8.8 and 50 mM NaCl). The extraction was performed three times until no additional protein was solubilized. Forty mL of Ni-NTA resin (Qiagen) was then added to the combined extract. The resin was first washed with pH 8.8 buffer followed by pH 8.8 buffer containing 50 mM imidazole. The R347K TNFR DD protein was then eluted with 400 mM imidazole in pH 8.8 buffer. DTT was added immediately to the eluate to a final concentration of 10 mM. The eluted protein was pooled and dialyzed against buffer containing 50 mM sodium acetate pH 4.0-4.2 and 10 mM DTT overnight. The protein was then concentrated using centriprep (YM-10, Amicon) to about 6 mg/ml and applied to G3000SW (TOSOHASS) column pre-equilibrated with 10 mM sodium acetate pH 4.0 and 10 mM DTT. The monomeric fraction was pooled and concentrated for NMR studies.

NMR data collection, complete assignments and secondary structure determination. All spectra were recorded at 35° C. on a Varian Unity Plus 600 spectrometer equipped with a triple-resonance ¹H/¹³C/¹⁵N probe and an actively shielded z-gradient pulsed field accessory. ¹H-¹⁵N HSQC and all HSQC-based ¹⁵N edited 3D experiments on ¹⁵N labeled and triple-resonance 3D experiments on ¹⁵N/¹³C double-labeled protein were recorded with the enhanced-sensitivity pulsed field gradient approach [23]. This approach provides coherence transfer selection both to improve sensitivity and to eliminate artifacts as well as for solvent suppression. The complete assignments (>95%) of the ¹H, ¹⁵N and ¹³C resonances were based on the following experiments: CBCA(CO)NNH, HNCACB, C(CC)TOCSY_NNH, H(CC)TOCSY_NNH, HAHB(CBCACO)NNH [23, 24] [Table 1S]. 2D ¹³C (methyl)-¹H HSQC and methyl relay experiments were used for auxiliary methyl assignments of valine, leucine and isoleucine residues[25, 26]. Some ambiguous resonances were further confirmed by analysis of the simultaneous ¹⁵N/¹³C-edited NOESY [27] experiment. R347K TNFR-1 DD is a proline rich protein, which contains several peptidyl-prolyl (Cis/Trans) isomers. Most of the prolines were detected using a new experiment that connects sequential residues via their ¹⁵N nuclei [28]. 4D ¹³C, 1³C-edited NOESY [29] were carried out on a double labeled (¹⁵N/¹³C) sample in D₂O to assign overlapped γ-methyls of valine and the δ-methyls of leucine and isoleucine residues and also to identify some long-range NOEs between these methyls. Data sets were typically processed and displayed on an SGI workstation using the NMRDraw and NMRPipe [30] programs. A skewed 60° phase-shifted sine-bell function and a single zero-filling were used in each of the three dimensions prior to Fourier transformation. For triple-resonance 3D experiments, the time domain was extended by a factor of two using froward-backward linear prediction in the ¹⁵N (t2) dimension. For constant-time ¹H-¹³C correlation experiments, mirror image linear prediction was used prior to zero-filling to double the time domain data points [31]. The programs PIPP and STAPP [32] were used for data analysis and semi-automatic assignments [31].

The secondary structure of R347K TNFR-1 DD was based on characteristic NOEs involving the amide NH, H_{α} and H_{β} protons from ¹⁵N edited NOESY and simultaneous ¹⁵N/¹³C-edited NOESY spectra, three-bond J-coupling constants ³J_{NHα} from the 3D HNHA, slowly exchanging amide protons with D₂O, and C_{α} and C₆₂ secondary chemical shifts. It was determined that the secondary structure of R347K TNFR-1 DD is composed of six helices, ranging in length from 7-10 residues. Helix 3 (residues D353-L361) appears to be more flexible as evident by the increased amide exchange rates relative to other helical regions in the protein.

R347K TNFR-1 DD Structure Calculation. The NMR solution structure is based on inter-proton distance constraints converted from observed NOEs in both the ¹⁵N-edited NOESY [33, 34] and simultaneous ¹⁵N/¹³C-edited NOESY experiments. The NOEs were classified as either strong (1.8-2.7 A), medium (1.8-3.3 A) or weak (1.8-5.5 A) constraints. Upper distance limits for distances involving methyl protons and non-stereospecifically assigned methylene protons were corrected appropriately for center averaging [35], and an additional 0.5 A was added to upper distance limits for NOEs involving methyl protons [36, 37]. φ and ψ torsion angle constraints were obtained from the 3D HNHA experiment [38] and from ¹⁵N, Hα, C_{α}and C_{β} chemical shifts using the TALOS [39] program.

The structures were calculated using the hybrid distance geometry-dynamical simulated annealing method of Nilges et al. [40] with minor modifications [41] using the program XPLOR [42], adapted to incorporate pseudopotential secondary C_{α}/Cβ chemical shift restraints [43] and a conformational database potential [44, 45]. The target function that is minimized during restrained minimization and simulated annealing comprises only quadratic harmonic terms for covalent geometry and secondary C_{α}/C_{β} chemical shift restraints, square-well quadratic potentials for the experimental distance and torsion angle restraints, and a quartic van der Waals term for non-bonded contacts. All peptide bonds were constrained to be planar and trans. There were no hydrogen-bonding, electrostatic or 6-12 Lennard-Jones empirical potential energy terms in the target function.

The N- and C-terminal regions of the protein were unstructured and contain several peptidyl-prolyl (Cis/Trans) isomers therefore, they were not used in the structure calculations. The structure of R347K TNFR-1 DD encompassing residues P327-D413 was determined from a total of 1167 distance constraints comprising 264 intra-residue, 279 sequential, 247 medium, and 295 long range distance constraints, 82 hydrogen bond constraints and 112 torsion angles constrains comprised of 56 φand 56 ψdihedral constraints. The hydrogen bond constraints were based on the observation of slow exchanging NH protons in a D₂O solution monitored by an ¹H-⁵¹N HSQC spectrum.

The final ensemble of 20 structures contained no distance constraint violations greater than 0.25 Å and no torsion angle constraint violations greater than 3°. The NMR structures are well defined, as evident by the atomic r.m.s.d. of the 20 simulated annealing structures about the mean coordinate positions where the backbone of the helical regions and all atom in the helical region is 0.40 Å and 1.09 Å, respectively (Table 1). The R347K TNFR-1 DD NMR structure is consistent with a good quality structure based on PROCHECK and Ramachandran analysis [46, 47]. A Ramachandran plot of the minimized average structure shows a total of 88.5% of the residues are in the most favored region and 11.5 % in the additional allowed region. PROCHECK analysis indicates an overall G-factor of 0.01 and only 1 bad contacts.

Results and Discussion

Structure Determination. Historically, death domains have posed problems for NMR studies at physiological pH due to their tendency to self-associate and form large molecular weight aggregates. This is also the case for the Death Effector Domains (DED) and the Caspase Recruiting Domains (CARD) which are structurally related to the death domains. Therefore, structural studies of the death domain superfamily of proteins requires relatively low (<4) or high (>8) pH to minimize the natural tendency for the proteins to self-aggregate. In addition, single point mutants which disrupt self-association were necessary for the structural studies of the FADD DED [16] and in the current study of TNFR-1 DD.

The wild type TNFR-1 DD was very insoluble and not amenable to NMR structural studies over a wide pH range (pH 4-10), therefore mutant proteins were made to improve the solubility and stability of the protein. As previously described, the solubility and stability of the R347A mutant at high pH (pH=8.8) allowed for secondary structure determination and an insight into the topological fold of the protein (see, Example 1). Due to severe broadening of the amide proton peaks because of rapid exchange with water at high pH (pH=8.8), complete assignment and structure determination of the protein was hindered. Nevertheless, it was demonstrated that the majority of TNFR-DD forms α-helices, where the amide proton exchange is reduced by hydrogen bond formation. This allowed for the observation of backbone chemical shifts and the determination of sequential assignments in the helical regions using triple resonance experiments. The mutation of R347 to lysine allowed for NMR studies to be performed at low pH (pH=4.0) where the exchange with water is reduced. At pH=4.0, the solubility of the R347K TNFR-1 DD mutant was -1 mM and the protein ran predominantly as a monomer on a gel filtration column. Additionally, the protein appears folded by HSQC correlation experiment collected on a ¹⁵N labeled protein.

FIG. 6 is a summary of NMR derived data that summarize the secondary structural elements of R347K TNFR-1 DD. Sequential and medium and range NOE's: d_{NN}(i,i+1), d_{αN}(i,i+3) d_{αN}(i,i+4) and d_{αβ}(i,i+3) along with ΔC_{α} and ΔC_{β} secondary chemical shifts identify the 6 helical regions of the protein. Additionally, the experimentally measured coupling constants (³J_{HNHα}) and the hydrogen-deuterium exchange data correlate well with the α-helices with the exception of helix 3 which shows that the amides located in helix 3 exchange quickly with D₂O. The approximate lengths of the helices are from residues: A328 to N336, W342 to L349, D353 to L361, L367 to R380, L389 to D398, and G403 to L412. The N- and C-terminal regions are unstructured and both exhibit multiple isomers (cis-trans) due to the presence of six proline residues.

The structure of the R347K TNFR-1 DD is well defined by 1360 experimentally derived NMR restraints (Table 1) consisting of 1167 NOE distance constraints, 112 dihedral angle constraints and 82 hydrogen bond constraints. An overlay of the backbone atoms for the best 20 simulated annealing structures for the R347K TNFR-1 DD was generated (not shown). As evident from the best-fit superposition of the R347K TNFR-1 DD NMR structures, the backbone and sidechains for the core of the protein consisting of the six helical regions are well defined having an r.m.s.d. of 0.40 Å and 1.09 Å, respectively. A ribbon diagram of the C_{α} trace of the average minimized structure of R347K TNFR-1 DD was also generated (not shown). The structure consists of six anti-parallel amphipathic α-helices where the majority of the hydrophobic residues form an extensive network of interactions that constitute the core of the protein. Helices 1, 5, and 6 are aligned anti-parallel to one another and are aligned almost perpendicular to helices 2, 3, and 4. The alignment of helix 3 is slightly skewed from being anti-parallel to helices 2 and 4. The surface of the R347K TNFR-1 DD mutant is composed of several basic (R341, K343, K347, R348, R358, R365, R368, R379, R380, R381, R384, R385, R394, and R397) and several acidic (E335, E344, D353, E355, D357, E360, E369, E386, E390, D398, D400, E406, D407, E409 and E410) residues. One face of the R347K TNFR-1 DD surface contains an acidic patch consisting of residues from helix 1 (E335), helix 5 (E386, E390, D398) and helix 6 (E406, D407, E409, and E410). The other face of the protein contains several exposed basic residues from helix 4 (R365, R368, R379, R380, and R381), the loop between helix 4 and helix 5 (R384 and R385), and from helix 5 (R394 and R397). The structure also contains several surface exposed hydrophobic residues (y331, L340, L349, L351, L361, L367, Y372, A376, L391, M399, L402, and L412). These electrostatic patches on the surface of TNFR-1 DD is suggestive of potential protein binding sites.

Structural comparison of TNFR-1 DD mutants. Due to the poor solubility of the wild type TNFR-1 death domain, NMR structural studies were pursued on two different mutant proteins at two distinct pH values. The R347A mutant was studied at high pH (pH 8.8) where the protein is soluble to 1 mM (see, Example 1); whereas the R347K TNFR-1 DD described herein is soluble to 1 mM at pH=4.0. The mutation in both proteins occurs in helix 2 and although the solubilities of the R347A and R347K TNFR-1 DD proteins are drastically different, the secondary structure in this region appears unaffected. Additionally, the pattern of inter-helical long range NOEs is conserved when comparing the two TNFR-1 DD mutant proteins further supporting the observation that the structures of the two mutant proteins are very similar. The only notable difference between the R347A and R347K TNFR-1 DD proteins resides in the length of helix 3. The helix spans 5 residues (E353-D357) in the R347A mutant compared to 8 residues (D353-L361) in the R347K TNFR-1 DD. The observed difference in the length of helix 3 for the two mutants may be attributed to an apparent increase in the mobility of this helix relative to the other helical regions as suggested by the exchange data (FIG. 6). At the relatively high pH used for the analysis of the R347A mutant structure, the amide exchange rate with solvent is rapid compared to the conditions used for the R347K TNFR-1 DD mutant. Therefore, it is not surprising that the remainder of helix 3 was not observed in the R347A study (see, Example 1).

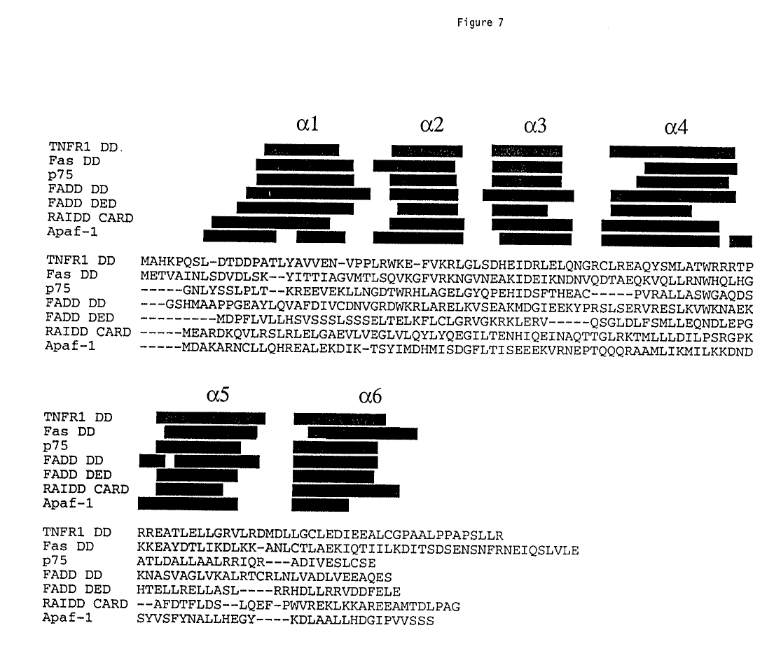

Structural comparison of TNFR-1 DD with other members of the death domain superfamily. The death domain superfamily of proteins includes the death domains, the death effector domains, and the CARD domains which are involved in signaling programmed cell death and homotypic interactions. Each known structure for the proteins in this superfamily contains a characteristic core of six anti-parallel α-helices [12-14, 16-19]. Typically there is ˜20% sequence identity between the various death domains [48] and less sequence homology when comparing the death domains to the death effector domains and CARD domains. All of the members in this class contain relatively short protein sequences (˜100 amino acids) that encompass the 6 α-helices. FIG. 7 presents a pseudo-structural and sequence alignment for members of the death domain superfamily of proteins.

A comparison of the R347K TNFR1 DD structure with the other known structures in the death domain superfamily has revealed that the overall fold for TNFR-1 DD is similar to other death domains. The superposition of the 6 α-helical regions of R347K TNFR-1 DD with the proteins in the death domain superfamily was generated (not shown). The observed deviations when comparing the R347K TNFR-1 DD structure to the other protein structures in the death domain superfamily reside in either helix length (FIG. 7) or helix orientation (not shown). A detailed analysis of the death domain structures revealed that the structure of p75 neurotrophin was the most similar to TNFR-1 DD having an r.m.s.d of 1.80 Å for the 68 C_{α} atoms composed of the helical core. The similarity to TNFR-1 DD is followed by the FAS DD and FADD DD having r.m.s.d's of 2.17 Å and 2.14 Å for the 70 C_{α} and 52 C_{α} atoms in the helical core, respectively. Comparison of TNFR-1 DD with the CARD domains resulted in r.m.s.d's of 1.95 Å and 2.17 Å for the 49 C_{α} and 53 C_{α} atoms in the core helical region for the RAIDD CARD and Apaf-1 structures, respectively. Overlay of R347K TNFR-1 DD mutant with the FADD DED resulted in a r.m.s.d. of 2.08 Å for the 56 C_{α} atoms in the helical regions of the proteins.

Although the structural superpostition of R347K TNFR-1 DD with the other proteins in the death domain superfamily yielded reasonable r.m.s.d's consistent with the similarity in the overall folds, the major structural difference between R347K TNFR-1 DD and the other death domain-like proteins resides in the relative orientations of helices 2, 3, and 4. In the R347K TNFR-1 DD structure, helices 2 and 4 are aligned parallel to one another and the orientation of helix 3 is almost perpendicular. P75 neurotrophin is the only member of the death domain superfamily that retains the orientation of helices 2, 3, and 4. The lengths of helices 2 and 3 are very similar among all the family members, however, helix 4 of FAS DD [12], p75 neurotrophin [14], and FADD DED [16] are much shorter (consisting of 11, 10, and 7 residues respectively) than the length of helix for R347K TNFR1-DD. Additionally, the length of helix 1 of R347K TNFR-1 DD consisting of 8 residues is much shorter than helix 1 of the FADD DD (14 residues) [13], the RAIDD CARD domain (13 residues) [17], and Apaf-1 (12 residues) [21].

Several of the proteins in the death domain superfamily (FAS DD [12], RAIDD CARD [17], and the FADD DD [13]) have charged surfaces and it is presumed that electrostatic interactions are the driving force for homotypic interactions. Recently, crystal structures of complexes formed between procaspase-9 with APAF-1 CARD domains [18] and the Pelle DD with the Tube DD [19] have shown that the surfaces involved in the homotypic interactions are charged and of opposite polarity. Nevertheless, the mode of interaction in both cases was shown to be driven by a network of hydrogen bond contacts as well as by van der Waals interactions instead of an electrostatic interaction as suggested by the protein surface. In the case of the TNFR-1 DD, the interaction between TNFR-1 DD and TRADD DD could be disrupted by salt (see, Example 1). This is consistent with the molecular surface for the NMR structure of the R347K TNFR-1 DD mutant which contains several charged residues containing an acidic patch on one face of the protein and several basic residues on the other face of the protein. Several of the charged residues are located in helices 2, and 3 which correlates well with the identified self-association and protein association sites on FAS DD [12] and FADD DD [13]. These observations suggest that the mode of interaction of TNFR-1 DD with TRADD DD may be primarily electrostatic in nature.

Mutagenesis studies set forth in Example 1 have shown that helix 2 and part of helices 3 and 4 contain important residues for both self-association and interaction with the TRADD DD. These regions of the TNRF-1 DD protein are also correlated with well-defined electrostatic regions on the proteins surface. The mutants that affect both self-association and interaction with the TRADD DD are located on two distinct faces of the protein. One face contains critical residues from helix 2 and part of helix 3 (K343A, E344A, R347A, R348A, and D353A). This is consistent with the previous observation that the surface formed by α-helices 2 and 3 has been implicated in the homo- and hetero-association of the death domains of FAS [12] and FADD [13]. A rotation of 180° about the z-axis highlights the other surface composed of key residues in helix 4 (E369A and Y372F). A similar result was seen with the Tube and Pelle death domains complex where α-helices 4 and 5 of the Pelle death domain interacts with α-helix 6 and the unique C-terminal tail of the Tube death domain [19].

The observation that mutations in two distinct surfaces of TNRF-1 DD disrupts both self-association and interaction with the TRADD DD is consistent with the proposed mechanism of TNFR-1 DD activity [4]. Since TNRF-1 DD trimer formation is a requirement for the recruitment of TRADD DD to the cell surface and the interaction of TRADD DD with TNRF-1 DD, the two surfaces identified on the TNRF-1 DD by site-directed mutagenesis correspond to the self-association binding sites (see, Example 1). This would imply that each self-association binding site interacts with one TNFR-1 DD in the trimer complex forming a triangular arrangement consistent with the X-ray structure of TNFR-1 [49]. Conversely, since mutagenesis of either site did not result in the elimination of TRADD DD association with TNRF-1 DD while maintaining the ability of TNFR-1 DD to self-associate, neither of the binding surfaces can be exclusively attributed to a TRADD DD binding site. Nevertheless, it is still plausible that the TRADD DD binding site overlaps with the TNFR-1 DD self-association site. In the case of the FAS [12] and FADD [13] death domains, mutagenesis data identified distinct binding sites associated with hetero-interaction implying a potential difference in the binding interaction between TNFR-1 DD and TRADD DD.

The surface of the R347K TNFR-1 DD structure also contains an acidic patch consisting of residues from helices 1 and 6, but mutation of E406A and E410A (of helix 6) had no effect on self-association or binding to TRADD DD (see, Example 1). The assembly of the TNFR-1 DD trimer may present this acidic patch as a potential binding site for an undetermined death domain in another signaling cascade. Additionally, the acidic patch on TNFR-1 DD may represent the binding site of an unidentified regulator of TNFR-1 activity that is potentially released upon trimer formation. A potential functional role for helix 6 in TNFR-1 DD would be consistent with the Tube and Pelle death domains complex where a-helix 6 from the Tube death domain comprises part of the interaction site [19].

The comparison of TNFR-1 DD with the other members of the death domain superfamily of proteins revealed that the major difference in the structures relative to TNFR-1 DD resides in the lengths and orientation of helices 2, 3, or 4. The location of the structural change between the death domains is consistent with mutagenesis studies that have identified critical residues for both self-association and binding to TRADD DD in these regions of the protein (see, Example 1). Particularly, the mutagenesis data identifies the two putative self-association binding sites for TNFR-1 DD. One site involving α2 and α3 are consistent with the identification of the homo-binding sites for FAS DD [12] and FADD DD [13]. The observed differences in the local structure and sequence between TNFR-1 DD and the other proteins in the death domain superfamily that correlate with the functional regions of the proteins may account for the TNFR1-DD's selectivity and specificity in the TNF signaling cascade while accounting for the similarity in the overall fold. The observations that the nature of the protein-protein interaction can vary greatly further supports the apparently versatile nature of the death domain fold.

TABLE I
Structural, Energetic Statistics and Atomic rms Differences
Structural and Energetic Statistics

| | | <SA> | (SA)r |
|---|---|---|---|
| rms deviations from exptl distance | | 0.0185 ± 0.001 | 0.0107 |
| restraints (Å) (1167) | | | |
| No. of distance restraint violations | | 0 | 0 |
| greater than 0.25 Å | | | |
| rms deviations from exptl dihedral | | 0.0510 ± 0.05 | 0.0655 |
| restraints (deg) (92) | | | |
| No. of dihedral restraint violations | | 0 | 0 |
| greater than 3 (deg) | | | |
| rms deviations from idealized | | | |
| covalent geometry | | | |
| bonds | (Å) | 0.0030 ± 0.0002 | 0.0015 |
| angles | (deg) | 0.409 ± 0.015 | 0.2860 |
| impropers | (deg) | 0.3732 ± 0.0123 | 0.2477 |

Energetics

| | | | |
|---|---|---|---|
| Eᵣₑₚₑₗ | (kcal mol⁻¹) | 19 ± 3 | 9.6 |
| E_{NOE} | (kcal mol⁻¹) | 20 ± 3 | 6.7 |
| E_{edih} | (kcal mol⁻¹) | 0.03 ± 0.05 | 0.03 |
| E_{bond} | (kcal mol⁻¹) | 13.6 ± 2.5 | 3.2 |
| Eᵤₘₚ | (kcal mol⁻¹) | 15.7 ± 1 | 6.9 |
| E_{ang} | (kcal mol⁻¹) | 67.1 ± 5 | 32 |

Non-hydrogen Atomic rms Differences (Å)

| | All residues^{a} | | Secondary structure^{b} | |
|---|---|---|---|---|
| | backbone | | backbone | |
| | atoms | all atoms | atoms | all atoms |
| <SA> vs SA | 0.62 ± 0.09 | 1.50 ± 0.11 | 0.40 ± 0.07 | 1.08 ± 0.08 |
| <SA> vs (SA)r | 0.71 ± 0.1 | 1.70 ± 0.23 | 0.44 ± 0.08 | 1.24 ± 0.10 |
| (SA)r vs SA | 0.34 | 0.81 | 0.21 | 0.59 |

The NMR structures are denoted as follows:
<SA > are the final 22 ensemble structures; SA is the mean structure obtained from averaging the cartesian coordinates of individual ensemble members; and (SA)r is the minimized average structure obtained by regularization of SA.
Eᵣₑₚₑₗ was calculated using a final force constant of 4.0 kcal mol⁻¹ Å⁻⁴ with van der Waals hard sphere radii scaled by 0.78.
E_{NOE} was calculated using a square-well potential with center-averaging and a force constant of 50 kcal mol⁻¹ Å⁻² .
E_{edih} was calculated using a force constant of 200 kcal mol⁻¹ rad⁻².
E_{bond}, E_{angle}, and Eᵢₘₚᵣₒₚₑᵣ were calculated using force constants of 1000 kcal mol^{-1 Å}⁻² , 500 kcal mol⁻¹ rad ⁻² and 500 kcal mol⁻¹ rad⁻², respectively.
^{a}In all atomic rms differences calculations, only the backbone atoms (N, C_{α}and C) are included in the least squared best fitting.
^{b}Core four helices.

TABLE 1S

| | HN | N | C_{α} | C_{β} | C_{γ} | C_{δ} | C′ | H_{α} | H_{β} | H_{γ} | H(others) | |
|---|---|---|---|---|---|---|---|---|---|---|---|---|
| M315 | | | | | | 24.19 | | | | | ε = 2.15 | |
| A316 | | | | | | | | | | | | |
| H317 | | | | | | | | | | | | |
| K318 | | | | | | | | | | | | |
| P319 | | | 63.3 | 32.15 | 27.49 | 50.71 | 174 | 4.55 | 2.41, 2.05 | 2.16 | 3.94, 3.76 | |
| Q320 | 8.61 | 120.9 | 56.06 | 29.78 | 34.02 | | 175.9 | 4.46 | 2.23, 2.13 | 2.44 | | |
| S321 | 8.38 | 117 | 58.19 | 63.99 | | | 174.4 | 4.59 | 3.99 | | | |
| L322 | 8.42 | 123.8 | 55.24 | 42.64 | 27.24 | 25.36, 23.71 | 176.7 | 4.53 | 1.74 | | 1 | |
| D323 | 8.56 | 121.2 | 54.32 | 40.95 | | | 176.5 | 4.82 | 2.91, 2.83 | | | |
| T324 | 8.15 | 113.7 | 62.38 | 69.54 | 21.82 | | 174.4 | 4.41 | 4.4 | 1.34 | | |
| D325 | 8.47 | 121.7 | 54.21 | 41.02 | | | 175.6 | 4.84 | 2.91, 2.83 | | | |
| D326 | 8.42 | 121.1 | 54.46 | 40.86 | | | | | | | | |
| P327 | | | 66.31 | 31.82 | 28.01 | | | 4.6 | 2.34, 2.18 | 2.63 | | |
| A328 | 8.34 | 118.6 | 55.39 | 18.57 | | | 181 | 4.38 | 1.63 | | | |
| T329 | 8 | 115.4 | 66.48 | 68.47 | 21.87 | | 175.5 | 4.13 | 4.48 | 1.29 | | |
| L330 | 8.13 | 118.8 | 58.4 | 40.3 | | 24.5 | | 4.13 | 2.20, 1.4 | | 0.92 | |
| Y331 | 8.86 | 118.1 | 63.31 | 37.75 | C_{δ}132.33 | C_{ε}117.99 | 177.9 | 4.38 | 3.12, 3.07 | H_{δ}7.02 | H_{ε}6.47 | |
| A332 | 7.97 | 120.9 | 55.17 | 18.24 | | | 180.4 | 4.27 | 1.68 | | | |
| V333 | 7.93 | 119.1 | 66.74 | 31.24 | 22.79, 23.63 | | 177.6 | 3.67 | 2.62 | 1.19, 0.89 | | |
| V334 | 8.06 | 118.7 | 66.43 | 31.78 | 22.81, 21.84 | | 177.1 | 3.61 | 2.41 | 1.14, 0.92 | | |
| E335 | 7.99 | 113.2 | 57.78 | 29.71 | 34.96 | | 177 | 4.12 | 2.12 | 2.51, 2.43 | | |
| N336 | 7.5 | 114.8 | 55.15 | 42.89 | | | 174.5 | 4.96 | 2.8, 2.62 | 7.24 | | |
| V337 | 7.67 | 123 | 61.55 | 33 | 22.72, 19.75 | | | 4.35 | 2.14 | 0.72, 0.55 | | |
| P338 | | | | 28 | 51.6 | | | | | 2.18, 1.87 | 4.33, 3.44 | |
| P339 | | | 65.93 | 32.18 | 27.66 | 51.06 | 179.8 | 4.38 | 2.50, 2.10 | 2.22, 2.17 | 4.05 | |
| L340 | 8.74 | 116.9 | 57.16 | 41.69 | 27.46 | 24.85, 23.27 | 178.3 | 4.43 | 1.90, 1.75 | | 1.08 | |
| R341 | 8.17 | 114.7 | 53.47 | 29.22 | 25.97 | 41.17 | 175.8 | 4.93 | 2.27, 1.62 | 1.9 | 3.34 | |
| W342 | 7.69 | 120.9 | 61.27 | 30.02 | C_{δ}126.39 | C_{ζ2}114.6 | 176.2 | 4.19 | 3.50, 3.44 | H_{δ}7.23 | 7.68 H_{H2}7.1 | H_{ζ3}6.93 H_{ε3}7.43 |
| | | | | | H_{ε1}127.8 | C_{HH2}124.0 | | | | H_{ε}9.61_{ζ2} | | |
| | | | | | | C_{ζ3}121.7 | | | | | | |
| | | | | | | C_{ε1}121.1 | | | | | | |
| K343 | 8.78 | 117.5 | 60.68 | 31.52 | 26.51 | 29.59, 42.25 | 177.7 | 3.87 | 1.99, 1.90 | 1.69, 1.46 | 3.07 | |
| E344 | 8.08 | 119 | 59.04 | 29.27 | 35.97 | | 177.6 | 3.91 | 2.19, 2.10 | 2.63 | | |
| F345 | 7.98 | 119.9 | 60.59 | 38.96 | C_{δ}132.06 | C_{ε}128.13 | 175.9 | 4.27 | 3.06, 2.75 | H_{δ}6.51 | H_{ε}6.76 | H_{ζ}6.76 |
| | | | | | C₆₈ 129.64 | | | | | | | |
| V346 | 7.96 | 110.3 | 65.01 | 30.75 | 24.00, 20.14 | | 178.8 | 3.28 | 1.91 | 0.92, 0.44 | | |
| K347 | 8.18 | 123.2 | 59.15 | 31.11 | 25.22 | | 181.8 | 4.44 | 1.93 | | | |
| R348 | 8.25 | 122.1 | 59.22 | 30.23 | 29.73 | 43.6 | 178.6 | 4.05 | 2 | 1.79 | 3.34 | |
| L349 | 7.29 | 118.2 | 55.43 | 40.72 | | 24.22, 21.31 | 177.2 | 3.93 | 1.30, 0.90 | −0.23 | −0.61 | |
| G350 | 7.59 | 104.6 | 45.16 | | | | 175.1 | 4.44, 3.75 | | | | |
| L351 | 7.68 | 122.8 | 55.71 | 41.66 | 29.43 | | 176.3 | 4.54 | 1.57, 1.50 | 1.75 | 0.92 | |
| S352 | 8.94 | 117.8 | 56.78 | 65.62 | | | 175.2 | 4.66 | 4.43, 4.15 | | | |
| D353 | 8.92 | 121.4 | 57.89 | 40.26 | | | 178 | 4.43 | 2.79, 2.66 | | | |
| H354 | 8.49 | 115.6 | 58.27 | 28.42 | | | 177 | 4.6 | 3.35 | | | |
| E355 | 7.95 | 120.3 | 59.12 | 30.31 | 37.13 | | 178.1 | 4.19 | 2.07 | 2.46 | | |
| I356 | 8.22 | 120.1 | 65.76 | 38.9 | 29.37 | 17.22, 14.77 | 177.5 | 3.72 | 2.05 | 1.86 | 1.07 | 0.83 |
| D357 | 8.3 | 118.8 | 56.79 | 40.04 | | | 178.2 | 4.5 | 2.86 | | | |
| R358 | 7.91 | 119 | 58.74 | 30.12 | 27.11 | 43.38 | 178.4 | 4.18 | 1.99 | 1.73 | 3.29 | |
| L359 | 8.13 | 120 | 57.35 | 41.84 | 27.57 | 26.03, 23.11 | 179.3 | 4.25 | 2.02, 1.63 | | 1 | |
| E360 | 8.39 | 119.5 | 58.77 | 28.87 | 35.09 | | 177.8 | | 2.26, 2.18 | 2.63, 2.33 | | |
| L361 | 7.97 | 120.2 | 56.79 | 42.18 | 27.11 | 24.99, 23.68 | 179.1 | 4.33 | 1.90, 1.82 | | 1.01 | |
| Q362 | 8.18 | 117.9 | 57.08 | 29.44 | 34.09 | | 176.2 | 4.3 | 2.22 | 2.58, 2.51 | | |
| N363 | 7.91 | 117.3 | 53.51 | 39.92 | | | 175.5 | 4.98 | 3.04, 2.77 | | | |
| G364 | 8.12 | 108.3 | 46.92 | | | | 174.8 | 4.00, 4.09 | | | | |
| R365 | 8.36 | 120 | 56.95 | 30.22 | 27.32 | 43.44 | 176.4 | 4.38 | 2 | 1.79 | 3.33 | |
| C366 | 8.12 | 119.2 | 58.09 | 27.55 | | | 174.7 | 4.68 | 3.13 | | | |
| L367 | 8.46 | 128.1 | 58.13 | 41.69 | 26.86 | 24.98, 23.54 | 178.1 | 4.22 | 1.76 | 1.54 | 0.94 | |
| R368 | 8.52 | 118.5 | 59.35 | 30.26 | 27.65 | 43.53 | 178.6 | 4.25 | 1.98, 179 | | 3.22 | |
| E369 | 7.7 | 117.4 | 58.86 | 28.93 | 36.04 | | 179.4 | 4.48 | 2.28 | 2.57 | | |
| A370 | 8.12 | 124.5 | 55.44 | 18.45 | | | 180.3 | 4.28 | 1.66 | | | |
| Q371 | 8.79 | 118.6 | 60.1 | 30.16 | 34.53 | | 178.3 | 4.05 | 1.15 | 2.22, 0.80 | | |
| Y372 | 8.61 | 120 | 62.49 | 38.25 | C_{δ} = 132.48 | C_{ε} = 118.51 | 177.2 | 3.85 | 3.27, 2.99 | H_{δ} = 6.60 | H_{ε} = 6.78 | |
| S373 | 8.61 | 114.7 | 62.26 | 62.93 | | | 177.7 | 4.25 | 4.20, 4.27 | | | |
| M374 | 8.28 | 122.6 | 59.91 | 33.72 | 33.93 | 19.11 | 177.3 | 4.2 | 3.16, 2.92 | 2.18 | 2.43 | |
| L375 | 7.84 | 119 | 57.38 | 42.46 | 27.39 | 23.87 | 178.7 | 4.42 | 2.02, 1.80 | | 0.91 | |
| A376 | 9.26 | 123.3 | 55.34 | 17.46 | | | 180.2 | 3.96 | 1.31 | | | |
| T377 | 8.05 | 116.3 | 67.19 | 68.62 | 20.89 | | 176 | 3.98 | 4.56 | 1.31 | | |
| W378 | 7.81 | 122.4 | 61.58 | 27.61 | C_{δ}127.4 | C_{HH2}122.7 | 178.2 | 4.12 | 3.72, 3.18 | H_{δ}7.2 | 7.43 H_{H2}7.1 | H_{ζ3}6.88 H_{ε3}7.93 |
| | | | | | H_{ε1}130.6 | C_{ζ3}119.5 | | | | H_{ε}10.01_{ζ2} | | |
| | | | | | C_{ζ2}115.0 | C_{ε3}120.8 | | | | | | |
| R379 | 9.06 | 122 | 57.78 | 32.15 | | | 178.9 | 4.32 | 2.20, 2.01 | | | |
| R380 | 7.99 | 118 | 58.41 | 30.56 | 28.02 | 43.62 | 177.3 | 4.22 | 2.01 | 1.97 | | |
| R381 | 7.34 | 116.1 | 55.83 | 30.71 | 26.04 | 44.08 | 175.4 | 4.54 | 1.91 | 2.01, 1.74 | 3.2 | |
| T382 | 7.15 | 120.2 | 60.53 | 69.8 | 20.63 | | | 4.52 | 3.28 | 1.08 | | |
| P383 | | | 63.71 | 32.52 | 27.51 | 51.42 | | 4.38 | 2.46, 2.14 | 1.92 | 4.03, 3.76 | |
| R384 | 8.55 | 123.5 | 57.28 | 29.8 | | | | 4.18 | 1.94 | | | |
| R385 | | | 56.24 | 30.7 | 27.32 | 43.4 | 176.1 | 4.33 | 1.81 | 1.65 | 3.27 | |
| E386 | 8.39 | 118.6 | 55.26 | 29.18 | | | | 4.35 | | | | |
| A387 | | | | | | | | | | | | |
| T388 | | | 61.93 | 69.77 | 22 | | 176 | 4.38 | | 1.8 | | |
| L389 | 9.36 | 118.9 | 57.91 | 41.19 | | 24.09 | 179.4 | 4.15 | 1.82 | | 0.93 | |
| E390 | 7.37 | 119.8 | 59.28 | 29.38 | 35.64 | | 178.8 | 4.25 | 2.3 | 2.47 | | |
| L391 | 7.79 | 120.1 | 58.09 | 42.32 | | 25.83 | 179.9 | 4.28 | 2.25, 1.96 | | 1.17 | |
| L392 | 8.55 | 116.9 | 57.79 | 41.66 | 27.09 | 27.35, 23.72 | 179.1 | 4.02 | 1.90, 1.40 | 2.10? | 0.92, 0.43 | |
| G393 | 8.51 | 106.4 | 47.87 | | | | 175.3 | 4.09, 3.84 | | | | |
| R394 | 7.47 | 121.3 | 59.54 | 30.17 | 27.4 | 43.84 | 178.1 | 4.12 | 2.13 | 1.86 | 3.36 | |
| V395 | 7.23 | 119 | 66.33 | 31.99 | 22.92, 22.33 | | 178.1 | 3.83 | 2.13 | 0.97, 0.97 | | |
| L396 | 8.46 | 118 | 58.34 | 40.65 | 26.5 | 26.57, 23.43 | 179.8 | 4.05 | 1.93, 1.41 | | 0.73, 0.73 | |
| R397 | 8.34 | 120.1 | 60.49 | 29.9 | 19.64 | 43.45 | 180.4 | 4.04 | 2.07, 1.95 | 1.64 | 3.32 | |
| D398 | 8.24 | 123.3 | 57.23 | 40.02 | | | 177.7 | 4.55 | 3.13, 2.78 | | | |
| M399 | 7.75 | 116.7 | 56.58 | 34.97 | 32.24 | 16.74 | 174.6 | 4.27 | 2.55, 1.93 | 2.83 | 2.16 | |
| D400 | 8.27 | 116.9 | 55.04 | 38.49 | | | 175.5 | 4.55 | 3.46, 2.83 | | | |
| L401 | 8.38 | 120.2 | 52.76 | 42.25 | 26.32 | 23.32 | 175.1 | 5.06 | 1.85, 1.74 | 2.81? | 1.05 | |
| L402 | 8.25 | 121.6 | 58.34 | 41.35 | 27.46 | 24.37 | 179.5 | 4.01 | 1.80, 1.72 | | 1.02 | |
| G403 | 8.83 | 108.1 | 47.02 | | | | 175.9 | 3.92 | | | | |
| C404 | 7.52 | 120.6 | 61.74 | 27.13 | | | 176.4 | 4.39 | 3.17 | | | |
| L405 | 7.69 | 119.7 | 58.05 | 41.36 | 25.92 | 26.1 | 177.9 | 4.11 | 2.30, 1.47 | | 1.04 | |
| E406 | 8.13 | 118.7 | 59.31 | 28.11 | 34.77 | | 178.4 | 4.14 | 2.32, 2.22 | 2.55 | | |
| D407 | 8.03 | 120.3 | 59.32 | 39.09 | | | 178.8 | 4.52 | 3.07, 2.82 | | | |
| I408 | 8.38 | 122.8 | 66.04 | 38.56 | 17.43, 15.21 | 177 | 3.55 | 2.13 | | | 0.88 | |
| E409 | 8.5 | 117.5 | 59.8 | 28.13 | 34.47 | | 179.2 | 3.97 | 2.35, 2.17 | 2.72, 2.38 | | |
| E410 | 8.44 | 118.8 | 58.83 | 28.66 | 34.84 | | 178.5 | 4.2 | 2.25 | 2.67, 2.52 | | |
| A411 | 7.93 | 122.1 | 54.35 | 18.33 | | | 179.4 | 4.28 | 1.67 | | | |
| L412 | 7.72 | 116.5 | 55.73 | 42.94 | 26.62 | 22.64 | 178 | 4.46 | 1.93, 1.76 | | 0.99 | |
| C413 | 8.04 | 116.8 | 59.43 | 28.49 | | | 175.1 | 4.6 | 3.11 | | | |
| G414 | 8.11 | 110.2 | 44.98 | | | | | 4.25 | | | | |
| P415 | | | 63.7 | 32.05 | 27.3 | 50.08 | | 4.49 | 2.39, 2.1 | | 3.78 | |
| A416 | 8.29 | 123.2 | 52.23 | 19.22 | | | 177.3 | 4.42 | 1.49 | | | |
| A417 | 8.08 | 122.4 | 52.31 | 19.36 | | | 177.3 | 4.41 | 1.48 | | | |
| L418 | 8.02 | 122.2 | 52.69 | 42 | | | | | | | | |
| P419 | | | | | | | | | | | | |
| P420 | | | 62.76 | 31.97 | 27.51 | | | 4.54 | 2.13, 2.01 | | 2.36 | |
| A421 | 8.36 | 125.8 | 50.61 | 18.3 | | | | | | | | |
| P422 | | | 64.02 | 31.98 | 27.49 | 50.48 | 177.5 | 4.47 | 2.13, 2.03 | 2.42 | 3.85, 3.77 | |
| S423 | 8.28 | 114.4 | 59.02 | 63.27 | | | 175.2 | 4.43 | 3.99 | | | |
| L424 | 8.16 | 123.5 | 55.79 | 42.24 | 27.11 | 25.03, 23.61 | 177.5 | 4.4 | 1.74 | | 0.99 | |
| L425 | 7.94 | 121.1 | 55.5 | 42.19 | 27.04 | 25.02, 23.49 | 177.4 | 4.36 | 1.68 | | 0.97 | |
| R426 | 8.08 | 120.4 | 56.21 | 30.68 | | | | | | | | |

### References

1. Carswell, E. A., et al., An endotoxin-induced serum factor that causes necrosis of tumors. Proc Natl Acad Sci USA, 1975. 72: p. 3666-3670.

2. Beuder, B. and A. Cerami, The biology of cachectin/TNF—a primary mediator of the host response. Annu Rev Immunol, 1989. 7: p. 625-655.

3. Smith, C. A., et al., A receptor for tumor necrosis factor defines an unusual family of cellular and viral proteins. Science, 1990. 48: p. 1019-1023.

4. Smith, C. A., T. Farrah, and R. G. Goodwin, The TNF receptor superfamily of cellular and viral proteins: activation, costimulation, and death. Cell, 1994. 76: p. 959-962.

5. Tartaglia, L. A. and D. V. Goeddel, Two TNF receptors. Immunol Today, 1992. 13: p. 151-153.

6. Vandenabeele, P., et al., Two tumor necrosis factor receptors: structure and function. Trends Cell Biol, 1995. 5: p. 392-399.

7. Tartaglia, L. A., et al., A novel domain within the 55 kd TNF receptor signals cell death. Cell, 1993. 74: p. 845-853.

8. Boldin, M. P., et al., Self-association of the “death domains” of the p55 tumor necrosis factor (TNF) receptor and Fas/APO1 prompts signaling for TNF and Fas/APO1 effects. J. Biol. Chem., 1995.270: p. 387-391.

9. Grell, M., et al., TR60 and TR80 tumor necrosis factor (TNF)-receptors can independently mediate cytolysis. Lymphokine Cytokine Res, 1993. 12(3): p. 143-8.

10. Tartaglia, L. A., et al., Tumor necrosis factor's cytotoxic activity is signaled by the p55 TNF receptor. Cell, 1993. 73(2): p. 213-6.

11. Hsu, H., et al., TRADD-TRAF2 and TRADD-FADD interactions define two distinct TNF receptor 1 signal transduction pathways. Cell, 1996. 84(2): p. 299-308.

12. Huang, B., et al., NMR structure and mutagenesis of the Fas (APO-1/CD95) death domain. Nature, 1996. 384: p. 638-641.

13. Jeong, E.-J., et al., The solution structure of FADD Death Domain. J. Biol. Chem., 1999. 274: p. 16337-16342.

14. Liepinsh, E., et al., NMR structure of the death domain of the p75 neurotrophin receptor. EMBO J., 1997. 16: p. 4999-5005.

15. Hofmann, K., P. Bucher, and J. Tschopp, The CARD domain: a new apoptotic signalling motif. Trends Biochem. Sci., 1997. 22: p. 155-6.

16. Eberstadt, M., et al., NMR structure and mutagenesis of the FADD (Mort1) death-effector domain. Nature, 1998. 392: p. 941-945.

17. Chou, J. J., et al., Solution structure of the RAIDD CARD and model for CARD/CARD interaction in caspase-2 and caspase-9 recruitment. Cell, 1998. 94(2): p. 171-80.

18. Qin, H., et al., Structural basis of procaspase-9 recruitment by the apoptotic protease-activating factor1. Nature, 1999. 399: p. 549-557.

19. Xiao, T., et al., Three-Dimensional Structure of a Complex between the Death Domains of Pelle and Tube. Cell, 1999. 99: p. 545-555.

20. Day, C. L., et al., Solution structure and mutagenesis of the caspase recruitment domain (CARD) from Apaf-1. Cell Death Differ., 1999. 6: p. 1125-32.

21. Zhou, P., et al.,

Solution structure of Apaf-1 CARD and its interaction with caspase-9 CARD: a structural basis for specific adaptor/caspase interaction. Proc Natl Acad Sci USA., 1999. 96: p. 11265-70.

22. Vaughn, D. E., et al., Crystal Structure of Apaf-1 Caspase Recruitment Domain: An α-Helical Greek Key Fold for Apoptotic Signaling. Journal of Molecular Biology, 1999.296: p. 937-947.

23. Kay, L. E., Pulsed field gradient multi-dimensional NMR methods for the study of protein structure and dynamics in solution. Prog Biophys Mol Biol, 1995. 63: p. 277-99.

24. Muhandiram, D. R. and L. E. Kay, Gradient enhanced triple-resonance three dimensional NMR experiments with improved sensitivity. J. Magn. Reson., 1994.103: p. 203-216.

25. Grzesiek, S., G. W. Vuister, and A. Bax, A simple and sensitive experiment for measurement of JCC couplings between backbone carbonyl and methyl carbons in isotopically enriched proteins. J Biomol NMR, 1993. 3(4): p. 487-93.

26. Bax, A., Max, D., and Zax, D., Journal of the american chemical society, 1992. 114: p. 6923-6925.

27. Pascal, S. M., et al., Nuclear magnetic resonance structure of an SH2 domain of phospholipase C-gamma 1 completed with a high affinity binding peptide. J. Magn. Reson., 1994. 103: p. 197-201.

28. Kanelis, V., et al., NMR studies of tandem WW domains of Nedd4 in complex with a PY motif-containing region of the epithelial sodium channel. Biochem Cell Biol, 1998. 76: p. 341-350.

29. Xu, G. Y., et al., Solution structure of a cellulose-binding domain from Cellulomonas fimi by nuclear magnetic resonance spectroscopy. Biochemistry, 1995. 34: p. 6993-7003.

30. Delaglio, F., et al., NMRPipe: a multidimensional spectral processing system based on UNIX pipes. J Biomol NMR, 1995. 6: p. 277-93.

31. Zhu, G.a.B., A., Journal of Magnetic Resonance, 1992. 100(202-207).

32. Garrett, D. S., et al., A common sense approach to peak picking in two-, three-, and four dimensional spectra using automatic computer analysis of contour diagrams. J. Magn. Reson., 1991. 95: p. 214-220.

33. Marion, D., et al., Overcoming the overlap problem in the assignment of 1H NMR spectra of larger proteins by use of three-dimensional heteronuclear 1H-15N Hartmann-Hahn-multiple quantum coherence and nuclear Overhauser-multiple quantum coherence spectroscopy: application to interleukin 1 beta. Biochemistry, 1989. 28(15): p. 6150-6.

34. Zuiderweg, E. R. and S. W. Fesik, Heteronuclear three-dimensional NMR spectroscopy of the inflammatory protein C5a. Biochemistry, 1989. 28(6): p. 2387-91.

35. Wuthrich, K., M. Billeter, and W. Braun, Pseudo-structuresfor the 20 common amino acids for use in studies of protein conformations by measurements of intramolecular proton-proton distance constraints with nuclear magnetic resonance. J Mol Biol, 1983. 169(4): p. 949-61.

36. Clore, G. M., et al., Three-dimensional structure of potato carboxypeptidase inhibitor in solution. A study using nuclear magnetic resonance, distance geometry, and restrained molecular dynamics. Biochemistry, 1987. 26(24): p. 8012-23.

37. Wagner, G., et al., Protein structures in solution by nuclear magnetic resonance and distance geometry. The polypeptide fold of the basic pancreatic trypsin inhibitor determined using two different algorithms, DISGEO and DISMAN. J Mol Biol, 1987. 196(3): p. 611-39.

38. Garrett, D. S., et al., The impact of direct refinement against three-bond HN-CaH coupling constants on protein structure determination by NMR. J. Magn. Reson., 1994. 104: p. 99-103.

39. Comilescu, G., F. Delaglio, and A. Bax, Protein backbone angle restraints from searching a database for chemical shift and sequence homology. J Biomol NMR, 1999. 13(3): p. 289-302.

40. Nilges, M., et al., Determination of three-dimensional structures of proteins by simulated annealing with interproton distance restraints. Application to crambin, potato carboxpeptidase inhibitor and barley serine proteinase inhibitor 2. Protein Eng, 1988. 2(1): p. 27-38.

41. Clore, G. M., et al., Three-dimensional structure of interleukin 8 in solution. Biochemistry, 1990. 29(7): p. 1689-96.

42. Brunger, A. T., X-PLOR Version 3.1 Manual. 1993, Yale University, New Haven Conn.

43. Kuszewski, J., et al., The impact of direct refinement against 13C alpha and 13C beta chemical shifts on protein structure determination by NMR. J Magn Reson B, 1995. 106(1): p. 92-6.

44. Kuszewski, J., A. M. Gronenborn, and G. M. Clore, Improving the quality of NMR and crystallographic protein structures by means of a conformational database potential derived from structure databases. Protein Sci, 1996. 5(6): p. 1067-80.

45. Kuszewski, J., A. M. Gronenbom, and G. M. Clore, Improvements and extensions in the conformational database potential for the refinement of NMR and X-ray structures of proteins and nucleic acids. J Magn Reson, 1997. 125(1): p. 171-7.

46. Laskowski, R. A., et al., AQUA and PROCHECK-NMR: programs for checking the quality of protein structures solved by NMR. J Biomol NMR, 1996. 8(4): p. 477-86.

47. Laskowski, R., et al., J. Appl. Cryst., 1993. 26: p. 283-291.

48. Feinstein, E., et al., The death domain: a module shared by proteins with diverse cellular functions. Trends Biochem Sci, 1995. 20: p. 342-44.

49. Eck, M. J., et al., Crystallization of trimeric recombinant human tumor necrosis factor (cachectin). J Biol Chem, 1988. 263(26): p. 12816-9.

50. Kay, L. E. & Bax, A., New methods for the measurement of NH-CαH coupling constants in 15N labeled proteins. J Magn Reson, 1990. 86: 110-126.

51. Hsu, et al., The TNF receptor-1-associated protein TRADD signals cell death and NF-kappa B activation. Cell, 1995. 81: 495-504.

52. Vandevoorde, V., et al., Induced expression of trimerized intracellular domains of the human tumor necrosis factor (TNF) p55 receptor elicits TNF effects. J Cell Biol, 1997. 137: 1627-1638.

53. Jiang, et al., Prevention of constitutive TNF receptor 1 signaling by silencer of death domains. Science, 1999. 283: 543-546.

54. Sali, et al., Evaluation of comparative protein modeling by MODELLER. Proteins, 1995. 23: 318-326.

55. Sanchez, R. & Sali, A., Evaluation of comparative protein structure modeling by MODELLER-3. Proteins: Struct., Funct., Genet., 1998. Suppl. 1: 50-58.

All publications mentioned herein above, whether to issued patents, pending applications, published articles, or otherwise, are hereby incorporated by reference in their entirety. While the foregoing invention has been described in some detail for purposes of clarity and understanding, it will be appreciated by one skilled in the art from a reading of the disclosure that various changes in form and detail can be made without departing from the true scope of the invention in the appended claims.

What is claimed is:

1. A solution comprising a tumor necrosis factor receptor 1 death domain (TNFR-1 DD).

2. The solution of claim 1, wherein the TNFR-1 DD comprises amino acid residues 316-425 of FIG. 6.

3. The solution of claim 2, wherein the TNFR-1 DD is either unlabeled, ¹⁵N enriched or ¹⁵N,¹³C enriched.

4. The solution of claim 1, wherein the secondary structure of TNFR-1 DD comprises six alpha helices.

5. The solution of claim 4, wherein α1 comprises amino acid residues A328-N336 of TNFR-1 DD, α2 comprises amino acid residues W342-L349 of TNFR-1 DD, α3 comprises amino acid residues P353-L361 of TNFR-1 DD, α4 comprises amino acid residues L367-R380 of TNFR-1 DD, α5 comprises amino acid residues L389-D398 of TNFR-1 DD and α6 comprises amino acid residues G403-L412 of TNFR-1 DD.

6. The solution of claim 5, wherein TNFR-1 DD has the structure defined by the relative structural coordinates according to FIG. 8, ± a root mean square deviation from the conserved backbone atoms of said amino acids of not more than 1.5 Å.

7. An active site of a TRADD DD binding protein or peptide, wherein said active site is characterized by a three dimensional structure comprising the relative structural coordinates of amino acid residues K343, E344, R347, R348 and D353 according to FIG. 8, ± a root mean square deviation from the conserved backbone atoms of said amino acids of not more than 1.5 Å.

8. An active site of a TRADD DD binding protein or peptide, wherein said active site is characterized by a three dimensional structure comprising the relative structural coordinates of amino acid residues E369 and Y373 according to FIG. 8, ± a root mean square deviation from the conserved backbone atoms of said amino acids of not more than 1.5 Å.

9. An active site of a TRADD DD binding protein or peptide, wherein said active site is characterized by a three dimensional structure comprising the relative structural coordinates of amino acid residues E335, E386, E390, D398, E406, D407, E409 and E410 according to FIG. 8, ± a root mean square deviation from the conserved backbone atoms of said amino acids of not more than 1.5 Å.

10. An active site of a TRADD DD binding protein or peptide, wherein said active site is characterized by a three dimensional structure comprising the relative structural coordinates of amino acid residues R358, R365, R368, R379, R380, R381, R384, R385, R394 and R397 according to FIG. 8, ± a root mean square deviation from the conserved backbone atoms of said amino acids of not more than 1.5 Å.

11. An agent which binds to the active site of claim 7, wherein said agent is an inhibitor of TNFR-1 DD function.

12. An agent which binds to the active site of claim 8, wherein said agent is an inhibitor of TNFR-1 DD function.

13. An agent which binds to the active site of claim 9, wherein said agent is an inhibitor of TNFR-1 DD function.

14. An agent which binds to the active site of claim 10, wherein said agent is an inhibitor of TNFR-1 DD function.

15. A method for identifying an agent that interacts with TNFR-1 DD, comprising the steps of:
(a) determining an active site of TNFR-1 DD from a three dimensional structure of TNFR-1 DD; and
(b) performing computer fitting analysis to identify an agent which interacts with said active site.

16. The method of claim 15, wherein the active site is determined from the three dimensional structure defined by the structural coordinates set forth in FIG. 8, ± a root mean square deviation from the conserved backbone atoms of said amino acids of not more than 1.5 Å.

17. The method of claim 15, wherein the active site is characterized by a three dimensional structure comprising the relative structural coordinates of amino acid residues K343, E344, R347, R348 and D353 according to FIG. 8, ± a root mean square deviation from the conserved backbone atoms of said amino acids of not more than 1.5 Å.

18. The method of claim 15, wherein the active site is characterized by a three dimensional structure comprising the relative structural coordinates of amino acid residues E369 and Y373 according to FIG. 8, ± a root mean square deviation from the conserved backbone atoms of said amino acids of not more than 1.5 Å.

19. The method of claim 15, wherein the active site is characterized by a three dimensional structure comprising the relative structural coordinates of amino acid residues E335, E386, E390, D398, E406, D407, E409 and E410 according to FIG. 8, ± a root mean square deviation from the conserved backbone atoms of said amino acids of not more than 1.5 Å.

20. The method of claim 15, wherein the active site is characterized by a three dimensional structure comprising the relative structural coordinates of amino acid residues R358, R365, R368, R379, R380, R381, R384, R385, R394 and R397 according to FIG. 8, ± a root mean square deviation from the conserved backbone atoms of said amino acids of not more than 1.5 Å.

21. The method of claim 15, further comprising contacting the identified agent with TNFR-1 DD in order to determine the effect the agent has on TNFR-1 DD.

22. The method of claim 21, wherein the agent is an inhibitor of TNFR-1 DD.

23. The method of claim 15, further comprising contacting the identified agent with TNFR-1 DD in the presence of a TNFR-1 DD binding molecule, and determining the effect the agent has on binding between TNFR-1 DD and the TNFR-1 DD binding molecule.

24. An agent identified by the method of claim 15.

25. A method for identifying a potential inhibitor of TNFR-1 DD, comprising the steps of:
(a) generating a three dimensional model of TNFR-1 DD using the relative structural coordinates of the amino acids of FIG. 8, ± a root mean square deviation from the conserved backbone atoms of said amino acids of not more than 1.5 Å;
(b) employing said three-dimensional model to design or select a potential inhibitor; and
(c) synthesizing or obtaining said potential inhibitor.

26. The method according to claim 25, wherein the potential inhibitor is designed de novo.

27. The method according to claim 25, wherein the potential inhibitor is designed from a known inhibitor.

28. The method of claim 25, further comprising contacting the potential inhibitor with TNFR-1 DD in order to determine the effect the inhibitor has on TNFR-1 DD.

29. The method of claim 25, further comprising contacting the potential inhibitor with TNFR-1 DD in the presence of a TNFR-1 DD binding molecule, and determining the effect the potential inhibitor has on binding between TNFR-1 DD and the TNFR-1 DD binding molecule.

30. The method according to claim 25, wherein the step of employing the three dimensional structure to design or select the potential inhibitor comprises the steps of:
(a) identifying chemical entities or fragments capable of associating with TNFR-1 DD; and
(b) assembling the identified chemical entities or fragments into a single molecule to provide the structure of the potential inhibitor.

31. The method according to claim 30, wherein the potential inhibitor is designed de novo.

32. The method according to claim 30, wherein the potential inhibitor is designed from a known inhibitor.

33. The method of claim 30, further comprising contacting the potential inhibitor with TNFR-1 DD in order to determine the effect the inhibitor has on TNFR-1 DD.

34. The method of claim 30, further comprising contacting the potential inhibitor with TNFR-1 DD in the presence of a TNFR-1 DD binding molecule, and determining the effect the potential inhibitor has on binding between TNFR-1 DD and the TNFR-1 DD binding molecule.

35. An inhibitor identified or designed by the method of claim 25.

36. An inhibitor identified or designed by the method of claim 30.
